(19) United States
(12) Patent Application Publication
(10) Pub. No.: US 2009/0306039 A1
(43) Pub. Date: Dec. 10, 2009

(54) COMPOUNDS AND COMPOSITIONS AS ITPKB INHIBITORS

(76) Inventors: Shifeng Pan, San Diego, CA (US); Yi Liu, San Diego, CA (US); Yun Feng Xie, San Diego, CA (US); Dai Cheng, San Diego, CA (US); yongqin Wan, Irvine, CA (US); Dong Han, San Diego, CA (US); Yang Yang, San Diego, CA (US); Wenqi Gao, San Diego, CA (US); Jiqing Jiang, San Diego, CA (US); Badry Bursulaya, San Diego, CA (US); Philip Chamberlain, San Diego, CA (US); Donald Karanewsky, Escondido, CA (US); Xia Wang, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(21) Appl. No.: 12/374,481

(22) PCT Filed: Jul. 20, 2007
(86) PCT No.: PCT/US07/74048

§ 371 Date: Jul. 6, 2009

Related U.S. Application Data
(60) Provisional application No. 60/832,681, filed on Jul. 21, 2006.
(60) Provisional application No. 60/893,874, filed on Mar. 8, 2007.

(51) Int. Cl. A61K 31/496; C07D 403/06; C07D 401/14; C07D 403/14; C07D 413/14; A61K 31/506; A61K 31/551; A61K 31/5377

(52) U.S. Cl. 514/210.2; 544/371; 544/364; 544/295; 540/575; 544/121; 514/254.05; 514/253.09; 514/252.19; 514/218; 514/236.5

(57) ABSTRACT

The invention provides a novel class of compounds, pharmaceutical compositions comprising such compounds and methods of using such compounds to treat or prevent diseases or disorders associated with abnormal or deregulated B cell activities, particularly diseases or disorders that involve aberrant activation of inositol 1,4,5-trisphosphate 3-kinase B (ITPKb).

### CROSS-REFERENCE TO RELATED APPLICATIONS

This application claims the benefit of priority to U.S. Provisional Patent Application Number 60/832,681, filed 21 Jul. 2006 and U.S. Provisional Patent Application No. 60/893,874, filed 8 Mar. 2007. The full disclosures of these applications are incorporated herein by reference in their entirety and for all purposes.

### BACKGROUND OF THE INVENTION

1. Field of the Invention

The invention provides a novel class of compounds, pharmaceutical compositions comprising such compounds and methods of using such compounds to treat or prevent diseases or disorders associated with abnormal or deregulated B cell activities, particularly diseases or disorders that involve aberrant activation of inositol 1,4,5-trisphosphate 3-kinase B (ITPKb).

2. Background

The protein kinases represent a large family of proteins, which play a central role in the regulation of a wide variety of cellular processes and maintaining control over cellular function. A partial, non-limiting, list of these kinases include: non-protein substrate kinases such as IPTKb; receptor tyrosine kinases such as platelet-derived growth factor receptor kinase (PDGF-R), the nerve growth factor receptor, trkB, Met, and the fibroblast growth factor receptor, FGFR3; non-receptor tyrosine kinases such Abl and the fusion kinase BCR-Abl, Lck, Csk, Fes, Bmx and c-src; and serine/threonine kinases such as b-RAF, c-RAF, sgk, MAP kinases (e.g., MKK4, MKK6, etc.) and SAPK2α, SAPK2β and SAPK3. Aberrant kinase activity has been observed in many disease states including benign and malignant proliferative disorders as well as diseases resulting from inappropriate activation of the immune and nervous systems.

The novel compounds of this invention inhibit the activity of ITPKb and are, therefore, expected to be useful in the treatment of ITPKb-associated diseases.

### SUMMARY OF THE INVENTION

In one aspect, the present invention provides compounds of Formula I:

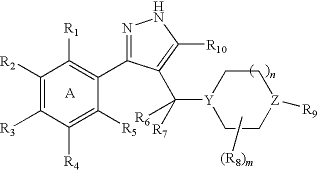

in which:

n is selected from 0, 1, 2 and 3;

m is selected from 0, 1, 2 and 3;

A can have up to 3 groups selected from —CR₁═, —CR₂═, —CR₃═, —CR₄═ and —CR₅═ replaced with N;

R₁, R₂, R₃, R₄ and R₅ are independently selected from hydrogen, hydroxy, halo, cyano, C₁₋₆alkyl, halo-substituted-C₁₋₆alkyl, hydroxy-substituted-C₁₋₆alkyl, cyano-substituted-C₁₋₆alkyl, C₃₋₈heterocycloalkyl-C₀₋₄alkyl, C₁₋₁₀heteroaryl-C₀₋₄alkyl, —XSO₂R₁₁, —XSO₂NR₁₁R₁₂, —XSO₂NR₁₁C(O)R₁₂, —XC(NR₁₁)NR₁₁OR₁₂, —XCR₁₁═NOR₁₂, —XC(O)R₁₁, —XC(O)OR₁₁, —XNR₁₁R₁₂, —XC(O)NR₁₁R₁₂, —XOC(O)NR₁₁R₁₂, —XNR₁₁C(O)NR₁₁R₁₂, —XNR₁₁XOR₁₂, —XN(XOR₁₂)₂, —XNR₁₁XC(O)OR₁₂, —XNR₁₁XNR₁₁R₁₂, —XNR₁₁XNR₁₁C(O)R₁₂, —XNR₁₁C(O)R₁₂; wherein each X is independently selected from a bond and C₁₋₄alkylene; each R₁₁ is selected from hydrogen and C₁₋₆alkyl; and R₁₂ is selected from hydrogen, C₁₋₆alkyl and C₆₋₁₀aryl; or R₁₁ and R₁₂ together with the nitrogen to which R₁₁ and R₁₂ are attached form a C₃₋₈heterocycloalkyl; wherein said heteroaryl or heterocycloalkyl of R₁, R₂, R₃, R₄ or R₅ is optionally substituted with 1 to 3 radicals independently selected from halo, hydroxy, cyano, C₁₋₆alkyl, halo-substituted-C₁₋₆alkyl, hydroxy-substituted-C₁₋₆alkyl, cyano-substituted-C₁₋₆alkyl and carboxy;

R₆ and R₇ are independently selected from hydrogen and C₁₋₃alkyl; or R₆ and R₇, together with the carbon to which they are both attached, forms C₃₋₇cycloalkyl;

R₈ is selected from C₁₋₆alkyl, halo-substituted-C₁₋₃alkyl, C₁₋₆ alkoxy, —CH₂OR₈ₐ, —COOR₈ₐ and C₂₋₆alkenyl; or two R₈ groups attached to different carbon atoms can combine to form an alkyl bridge; or two R₈ groups attached to the same carbon can form a C₃₋₈cycloalkyl group or a carbonyl group; wherein R₈ₐ is selected from hydrogen and C₁₋₆alkyl;

R₉ is selected from C₆₋₁₀aryl and C₁₋₁₀heteroaryl; wherein said aryl or heteroaryl of R₉ is optionally substituted with 1 to 3 radicals independently selected from halo, cyano, hydroxy, C₁₋₃alkyl, halo-substituted-C₁₋₃alkyl, cyano-substituted-C₁₋₃alkyl, hydroxy-substituted-C₁₋₃alkyl, —C(O)R₁₃, —C(O)NR₁₃R₁₄; wherein each R₁₃ and R₁₄ are independently selected from hydrogen and C₁₋₆alkyl;

R₁₀ is selected from hydrogen, C₁₋₆alkyl, —NR₁₅R₁₆, —NR₁₅C(O)R₁₆ and —C(O)NR₁₅R₁₆; wherein each R₁₅ and R₁₆ are independently selected from hydrogen, C₁₋₆alkyl, C₆₋₁₀aryl, C₁₋₁₀heteroaryl, C₃₋₁₂cycloalkyl and C₃₋₈heterocycloalkyl; wherein said aryl, heteroaryl, cycloalkyl and heterocycloalkyl can be optionally substituted with 1 to 3 radicals independently selected from halo, hydroxy, cyano, C₁₋₆alkyl, halo-substituted-C₁₋₆alkyl, C₁₋₆alkoxy and halo-substituted-C₁₋₆alkoxy;

y and Z are independently selected from CR₂₀ and N; wherein R₂₀ is selected from hydrogen and C₁₋₄alkyl; and the pharmaceutically acceptable salts thereof; with the proviso that compounds of Formula I do not include compounds of Formula II and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which inhibition of kinase activity, particularly ITPKb activity, can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which kinase activity, particularly ITPKb activity, contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group.

“Heteroaryl” means a saturated, unsaturated or partially saturated monocyclic ring containing 5 to 7 ring members selected from C, O, N and S″ includes, for example, pyridyl, indolyl, imidazolyl, pyrimidinyl, furanyl, oxazolyl, isoxazolyl, triazolyl, tetrazolyl, pyrazolyl, thienyl, morpholino, pyrrolidinyl, pyrrolidinyl-2-one, piperazinyl, piperidinyl, piperidinylone, etc.

“a bridged or fused bicyclic ring system containing 8 to 14 members selected from C, O, N and S (can be saturated, unsaturated or partially saturated) includes, for example, indazolyl, quinoxalinyl, quinolinyl, benzofuranyl, benzopyranyl, benzothiopyranyl, benzo[1,3]dioxole, benzo-imidazolyl, 1,4-dioxa-8-aza-spiro[4.5]dec-8-yl, etc.

“Compounds of Formula II” are compounds selected from 1-(2-ethoxyphenyl)-4-((3-(4-methoxyphenyl)-1H-pyrazol-4-yl)methyl)piperazine, 1-(2-methoxyphenyl)-4-((3-(4-methoxyphenyl)-1H-pyrazol-4-yl)methyl)piperazine, 1-(4-fluoro-phenyl)-4-((3-(4-methoxyphenyl)-1H-pyrazol-4-yl)methyl)piperazine, 1-(2-ethoxyphenyl)-4-((3-phenyl-1H-pyrazol-4-yl)methyl)piperazine, 1-(2-methoxyphenyl)-4-((3-phenyl-1H-pyrazol-4-yl)methyl)piperazine, 1-(2-ethoxyphenyl)-4-((3-phenyl-1H-pyrazol-4-yl)methyl)piperazine, 1-(5-(trifluoromethyl)pyridin-2-yl)-4-((3-(4-methoxyphenyl)-1H-pyrazol-4-yl)methyl)piperazine, 1-(3-chloro-5-(trifluoromethyl)pyridin-2-yl)-4-((3-(4-methoxyphenyl)-1H-pyrazol-4-yl)methyl)piperazine, 1-((3-(4-methoxyphenyl)-1H-pyrazol-4-yl)methyl)-4-(pyridin-2-yl)piperazine, 1-(3-chloro-5-(trifluoromethyl)pyridin-2-yl)-4-((3-(4-phenyl-1H-pyrazol-4-yl)methyl)piperazine, 1-((3-phenyl-1H-pyrazol-4-yl)methyl)-4-(pyridin-2-yl)piperazine, 1-(2-ethoxyphenyl)-4-((3-(4-fluorophenyl)-1H-pyrazol-4-yl)methyl)piperazine, 1-((3-(4-fluorophenyl)-1H-pyrazol-4-yl)methyl)-4-(2-methoxyphenyl)piperazine, 1-(4-(trifluoromethyl)pyridin-2-yl)-4-((3-(4-fluorophenyl)-1H-pyrazol-4-yl)methyl)piperazine, 1-(5-(trifluoromethyl)pyridin-2-yl)-4-((3-(4-fluorophenyl)-1H-pyrazol-4-yl)methyl)piperazine, 1-(3-chloro-5-(trifluoromethyl)pyridin-2-yl)-4-((3-(4-fluorophenyl)-1H-pyrazol-4-yl)methyl)piperazine, 1-((3-(4-fluorophenyl)-1H-pyrazol-4-yl)methyl)-4-(pyridin-2-yl)piperazine, and 1-(4-fluorophenyl)-4-((3-(4-fluorophenyl)-1H-pyrazol-4-yl)methyl)piperazine.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means the same as cycloalkyl above except that up to 3 ring carbons can be replaced with a group selected from C(O), NR₃₀, O, S(O)₀₋₂; wherein R₃₀ is selected from hydrogen and C₁₋₆alkyl. Heterocycloalkyl includes imidazolidine, pyrrolidine, piperidine, etc. C₃₋₈heterocycloalkyl-C₀₋₄alkyl, as used in this application to describe substituents R₁ to R₅, includes pyrrolidinyl-methyl, where the methyl is the point of attachment to ring A, for example.

“Halogen” (or halo) preferably represents chloro or fluoro, but may also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of kinase related disease, particularly IPTKb related diseases. For example, autoimmune diseases, particularly B cell associated diseases, are related to IPTKb. For example, rheumatoid arthritis, systemic lupus erythematosus (SLE), immune thrombocytopenic purpura (ITP) and hemolytic anemia.

In one embodiment, with reference to compounds of Formula I, n is selected from 1 and 2; m is selected from 0, 1 and 2; and A can have up to 3 groups selected from —CR₁═, —CR₂═, —CR₃═, —CR₄═ and —CR₅═ replaced with N.

In another embodiment, R₂, R₃ and R₄ are independently selected from hydrogen, hydroxy, halo, cyano, C₁₋₆alkyl, halo-substituted-C₁₋₆alkyl, hydroxy-substituted-C₁₋₆alkyl, cyano-substituted-C₁₋₆alkyl, C₃₋₈heterocycloalkyl-C₀₋₄alkyl, C₁₋₁₀heteroaryl-C₀₋₄alkyl, —XSO₂R₁₁, —XSO₂NR₁₁R₁₂, —XSO₂NR₁₁C(O)R₁₂, —XC(NR₁₁)NR₁₁OR₁₂, —XCR₁₁═NOR₁₂, —XC(O)R₁₁, —XC(O)OR₁₁, —XNR₁₁R₁₂, —XC(O)NR₁₁R₁₂, —XOC(O)NR₁₁R₁₂, —XNR₁₁C(O)NR₁₁R₁₂, —XNR₁₁XOR₁₂, —XN(XOR₁₂)₂, —XNR₁₁XC(O)OR₁₂, —XNR₁₁C(O)R₁₂; wherein each X is independently selected from a bond and C₁₋₄alkylene; each R₁₁ is selected from hydrogen and C₁₋₆alkyl; and R₁₂ is selected from hydrogen, C₁₋₆alkyl and C₆₋₁₀aryl; wherein said heteroaryl or heterocycloalkyl of R₁, R₂, R₃, R₄ or R₅ is optionally substituted with 1 to 3 radicals independently selected from halo, hydroxy, cyano, C₁₋₆alkyl, halo-substituted-C₁₋₆alkyl, hydroxy-substituted-C₁₋₆alkyl, cyano-substituted-C₁₋₆alkyl and carboxy.

In another embodiment, R₁, R₅, R₆ and R₇ are hydrogen; and R₈ is selected from C₁₋₂alkyl, halo-substituted-C₁₋₃alkyl, C₁₋₆ alkoxy, —CH₂OR₈ₐ, —COOR₈ₐ and C₂₋₆alkenyl; or two R₈ groups attached to different carbon atoms can combine to form an alkyl bridge; or two R₈ groups attached to the same carbon can form a C₃₋₈cycloalkyl group or a carbonyl group; wherein R₈ₐ is selected from hydrogen and C₁₋₆alkyl;

In another embodiment, R₉ is selected from C₆₋₁₀aryl and C₁₋₁₀heteroaryl; wherein said aryl or heteroaryl of R₉ is optionally substituted with 1 to 3 radicals independently selected from halo, cyano, hydroxy, C₁₋₃alkyl, halo-substituted-C₁₋₃alkyl, cyano-substituted-C₁₋₃alkyl, hydroxy-substituted-C₁₋₃alkyl, —C(O)R₁₃, —C(O)NR₁₃R₁₄; wherein each R₁₃ and R₁₄ are independently selected from hydrogen and C₁₋₆alkyl; and R₁₀ is hydrogen.

In another embodiment, Y is nitrogen; and A can have a group selected from —CR₁═, —CR₂═, —CR₃═, —CR₄═ and —CR₅═ replaced with nitrogen.

In another embodiment, R₂, R₃ and R₄ are independently selected from hydrogen, hydroxy, cyano, cyano-methyl, fluoro, chloro, bromo, iodo, amino-carbonyl, amino-carbonyl-methyl, tetrazolyl, amidino, methyl-carbonyl, 1-(hydroxy-imino)ethyl, amino-methyl, dimethyl-amino-methyl, N-ethylformamide, methyl-amino-carbonyl, dimethyl-amino, carboxy-methyl, methyl-amino-carboxy, ethyl-amino-carboxy, imidazolyl, pyrazolyl, 3-ethylureido, isopropyl-amino-carboxy, phenyl-amino-carboxy, hydroxy-carbonyl-methyl-amino, 2-hydroxy-ethoxy, 2-hydroxypropylamino, amino-carboxy, hydroxy-ethyl-amino, pyrrolidinyl substituted with carboxy, isoxazolyl, 2-hydroxy-methyl-pyrrolidin-1-yl, 3-hydroxypyrrolidin-1-yl, 3-hydroxyazetidin-1-yl, pyrrolyl optionally substituted with cyano, methyl-amino-sulfonyl, methyl-sulfonyl, methyl-carbonyl-amino-sulfonyl, carboxy, tetrazolyl, tetrazolyl-methyl, dihydroxyethyl-amino, oxazolyl, imidazolyl optionally substituted with methyl, pyrazolyl and 1,2,4-trazolyl.

In another embodiment, R₈ is selected from methyl, ethyl, methoxy-carbonyl, carboxy, trifluoromethyl and fluoromethyl; or two R₈ groups attached to the same carbon can form cyclopropyl; or two R₈ groups can combine to form a methyl, ethyl or propyl bridge such as, for example, a divalent radical of formula (a), (b) or (c), respectively:

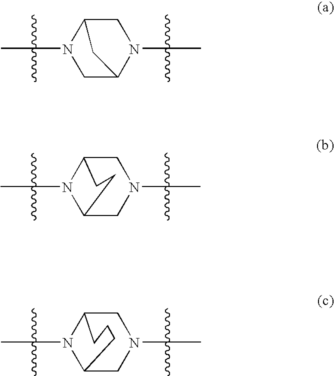

In another embodiment, R₉ is selected from phenyl, pyridinyl, pyrazinyl, pyrimidinyl and furo[3,2-c]pyridin-4-yl; wherein said phenyl, pyridinyl, pyrazinyl, pyrimidinyl or furo[3,2-c]pyridin-4-yl is optionally substituted with 1 to 3 radicals independently selected from trifluoromethyl, cyano, bromo, chloro, hydroxy-methyl, methyl-carbonyl, methyl, amino-carbonyl, nitro, iodo, fluoro, methoxy-carbonyl, hydroxy, amino, carboxy and methoxy.

In another embodiment are compounds of Formula I selected from 4-{4-[4-(5-Trifluoromethyl-pyridin-2-yl)-piperazin-1-ylmethyl]-1H-pyrazol-3-yl}-benzonitrile, Methyl-carbamic acid 4-{4-[3-methyl-4-(5-trifluoromethyl-pyridin-2-yl)-piperazin-1-ylmethyl]-1H-pyrazol-3-yl}-phenyl ester, 4-[3-(4-Imidazol-1-yl-phenyl)-1H-pyrazol-4-ylmethyl]-2-methyl-1-(5-trifluoromethyl-pyridin-2-yl)-piperazine, 4-[3-(6-Chloro-pyridin-3-yl)-1H-pyrazol-4-ylmethyl]-2-methyl-1-(5-trifluoromethyl-pyridin-2-yl)-piperazine, 1-[3-(4-Fluoro-phenyl)-1H-pyrazol-4-ylmethyl]-4-(4-trifluoromethyl-phenyl)-piperazine, 6-{4-[3-(4-Fluoro-phenyl)-1H-pyrazol-4-ylmethyl]-piperazin-1-yl}-nicotinonitrile, 1-(5-Bromo-pyridin-2-yl)-4-[3-(4-fluoro-phenyl)-1H-pyrazol-4-ylmethyl]-piperazine, 1-(5-Chloro-pyridin-2-yl)-4-[3-(4-fluoro-phenyl)-1H-pyrazol-4-ylmethyl]-piperazine, (6-{4-[3-(4-Fluoro-phenyl)-1H-pyrazol-4-ylmethyl]-piperazin-1-yl}-pyridin-3-yl)-methanol, 1-(6-{4-[3-(4-Fluoro-phenyl)-1H-pyrazol-4-ylmethyl]-piperazin-1-yl}-pyridin-3-yl)-ethanone, 1-(3,5-Dichloro-pyridin-4-yl)-4-[3-(4-fluoro-phenyl)-1H-pyrazol-4-ylmethyl]-piperazine, 4-[3-(4-Fluoro-phenyl)-1H-pyrazol-4-ylmethyl]-3,4,5,6-tetrahydro-2H-[1,2′]bipyrazinyl, 2-{4-[3-(4-Fluoro-phenyl)-1H-pyrazol-4-ylmethyl]-piperazin-1-yl}-nicotinonitrile, 1-(6-Chloro-pyridin-2-yl)-4-[3-(4-fluoro-phenyl)-1H-pyrazol-4-ylmethyl]-piperazine, 2-{4-[3-(4-Fluoro-phenyl)-1H-pyrazol-4-ylmethyl]-piperazin-1-yl}-4-trifluoromethyl-pyrimidine, 1-[3-(4-Fluoro-phenyl)-1H-pyrazol-4-ylmethyl]-4-(6-methyl-pyridin-2-yl)-piperazine, 2-{4-[3-(4-Fluoro-phenyl)-1H-pyrazol-4-ylmethyl]-piperazin-1-yl}-pyrimidine, 1-[3-(4-Fluoro-phenyl)-1H-pyrazol-4-ylmethyl]-4-(5-trifluoromethyl-pyridin-2-yl)-[1,4]diazepane, 1-[3-(4-Fluoro-phenyl)-1H-pyrazol-4-ylmethyl]-2,6-dimethyl-4-(5-trifluoromethyl-pyridin-2-yl)-piperazine, 1-[3-(4-Fluoro-phenyl)-1H-pyrazol-4-ylmethyl]-4-pyridin-2-yl-piperazine, 1-[3-(4-Fluoro-phenyl)-1H-pyrazol-4-ylmethyl]-4-(3-trifluoromethyl-pyridin-2-yl)-piperazine, 6-{4-[3-(4-Fluoro-phenyl)-1H-pyrazol-4-ylmethyl]-piperazin-1-yl}-nicotinamide, 4-{4-[3-(4-Fluoro-phenyl)-1H-pyrazol-4-ylmethyl]-piperazin-1-yl}-furo[3,2-c]pyridine, 1-[3-(4-Fluoro-phenyl)-1H-pyrazol-4-ylmethyl]-4-(5-nitro-pyridin-2-yl)-piperazine, 4-{4-[4-(5-Trifluoromethyl-pyridin-2-yl)-piperazin-1-ylmethyl]-1H-pyrazol-3-yl}-benzamide, 1-{3-[4-(1H-Tetrazol-5-yl)-phenyl]-1H-pyrazol-4-ylmethyl}-4-(5-trifluoromethyl-pyridin-2-yl)-piperazine, N-Hydroxy-4-{4-[4-(5-trifluoromethyl-pyridin-2-yl)-piperazin-1-ylmethyl]-1H-pyrazol-3-yl}-benzamidine, 1-(4-{4-[4-(5-Trifluoromethyl-pyridin-2-yl)-piperazin-1-ylmethyl]-1H-pyrazol-3-yl}-phenyl)-ethanone, 1-(4-{4-[4-(5-Trifluoromethyl-pyridin-2-yl)-piperazin-1-ylmethyl]-1H-pyrazol-3-yl}-phenyl)-ethanone oxime, 4-{4-[4-(5-Trifluoromethyl-pyridin-2-yl)-piperazin-1-ylmethyl]-1H-pyrazol-3-yl}-benzoic acid methyl ester, 1-[3-(4-Fluoro-phenyl)-1H-pyrazol-4-ylmethyl]-4-(5-iodo-pyridin-2-yl)-piperazine, 1-(4-Chloro-3-trifluoromethyl-phenyl)-4-[3-(4-fluoro-phenyl)-1H-pyrazol-4-ylmethyl]-piperazine, 1-[3-(4-Fluoro-phenyl)-1H-pyrazol-4-ylmethyl]-4-(3-trifluoromethyl-phenyl)-piperazine, 1-(4-Bromo-phenyl)-4-[3-(4-fluoro-phenyl)-1H-pyrazol-4-ylmethyl]-piperazine, 4-{4-[3-(4-Fluoro-phenyl)-1H-pyrazol-4-ylmethyl]-piperazin-1-yl}-phenol, 6-{4-[3-(4-Fluoro-phenyl)-1H-pyrazol-4-ylmethyl]-piperazin-1-yl}-pyridin-3-ylamine, 1-(3,4-Dimethyl-phenyl)-4-[3-(4-fluoro-phenyl)-1H-pyrazol-4-ylmethyl]-piperazine, 1-(2-Fluoro-phenyl)-4-[3-(4-fluoro-phenyl)-1H-pyrazol-4-ylmethyl]-piperazine, 6-{4-[3-(4-Fluoro-phenyl)-1H-pyrazol-4-ylmethyl]-piperazin-1-yl}-nicotinic acid, 4-[3-(4-Fluoro-phenyl)-1H-pyrazol-4-ylmethyl]-2-methyl-1-(5-trifluoromethyl-pyridin-2-yl)-piperazine, 1-[3-(4-Fluoro-phenyl)-1H-pyrazol-4-ylmethyl]-2-methyl-4-(5-trifluoromethyl-pyridin-2-yl)-piperazine, 1-[3-(4-Fluoro-phenyl)-1H-pyrazol-4-ylmethyl]-4-(5-methyl-pyridin-2-yl)-piperazine, 1-(3-Chloro-pyridin-2-yl)-4-[3-(4-fluoro-phenyl)-1H-pyrazol-4-ylmethyl]-piperazine, 2-{4-[3-(4-Fluoro-phenyl)-1H-pyrazol-4-ylmethyl]-piperazin-1-yl}-isonicotinonitrile, 2-Fluoro-5-{4-[4-(5-trifluoromethyl-pyridin-2-yl)-piperazin-1-ylmethyl]-1H-pyrazol-3-yl}-benzonitrile, 2-Fluoro-5-{4-[4-(5-trifluoromethyl-pyridin-2-yl)-piperazin-1-ylmethyl]-1H-pyrazol-3-yl}-benzamide, 4-{4-[4-(5-Trifluoromethyl-pyridin-2-yl)-[1,4]diazepan-1-ylmethyl]-1H-pyrazol-3-yl}-benzonitrile, 2-Fluoro-5-{4-[4-(5-trifluoromethyl-pyridin-2-yl)-piperazin-1-ylmethyl]-1H-pyrazol-3-yl}-benzylamine, (2-Fluoro-5-{4-[4-(5-trifluoromethyl-pyridin-2-yl)-piperazin-1-ylmethyl]-1H-pyrazol-3-yl}-benzyl)-dimethyl-amine, N-(2-Fluoro-5-{4-[4-(5-trifluoromethyl-pyridin-2-yl)-piperazin-1-ylmethyl]-1H-pyrazol-3-yl}-benzyl)-formamide, 1-(4-Chloro-phenyl)-4-[3-(4-fluoro-phenyl)-1H-pyrazol-4-ylmethyl]-piperazine, 1-[3-(4-Fluoro-phenyl)-1H-pyrazol-4-ylmethyl]-4-(4-methoxy-phenyl)-piperazine, 1-[3-(4-Fluoro-phenyl)-1H-pyrazol-4-ylmethyl]-4-p-tolyl-piperazine, 1-(3-Chloro-phenyl)-4-[3-(4-fluoro-phenyl)-1H-pyrazol-4-ylmethyl]-piperazine, 1-(2,4-Difluoro-phenyl)-4-[3-(4-fluoro-phenyl)-1H-pyrazol-4-ylmethyl]-piperazine, 1-(3,4-Dichloro-phenyl)-4-[3-(4-fluoro-phenyl)-1H-pyrazol-4-ylmethyl]-piperazine, 1-(2,3-Dichloro-phenyl)-4-[3-(4-fluoro-phenyl)-1H-pyrazol-4-ylmethyl]-piperazine, 1-(3,5-Dichloro-phenyl)-4-[3-(4-fluoro-phenyl)-1H-pyrazol-4-ylmethyl]-piperazine, 1-(2,3-Dimethyl-phenyl)-4-[3-(4-fluoro-phenyl)-1H-pyrazol-4-ylmethyl]-piperazine, 1-(2,4-Dimethyl-phenyl)-4-[3-(4-fluoro-phenyl)-1H-pyrazol-4-ylmethyl]-piperazine, 4-{4-[4-(5-Chloro-pyridin-2-yl)-piperazin-1-ylmethyl]-1H-pyrazol-3-yl}-benzonitrile, 2-{4-[3-(4-Cyano-phenyl)-1H-pyrazol-4-ylmethyl]-piperazin-1-yl}-isonicotinonitrile, 4-{4-[4-(4-Chloro-3-trifluoromethyl-phenyl)-piperazin-1-ylmethyl]-1H-pyrazol-3-yl}-benzonitrile, 4-{4-[4-(3,4-Dimethyl-phenyl)-piperazin-1-ylmethyl]-1H-pyrazol-3-yl}-benzonitrile, 1-[3-(4-Fluoro-phenyl)-1H-pyrazol-4-ylmethyl]-4-(4-methyl-pyridin-2-yl)-piperazine, 1-(2,4-Dichloro-phenyl)-4-[3-(4-fluoro-phenyl)-1H-pyrazol-4-ylmethyl]-piperazine, 1-(4-Chloro-2-fluoro-phenyl)-4-[3-(4-fluoro-phenyl)-1H-pyrazol-4-ylmethyl]-piperazine, 2-Cyano-5-{4-[4-(5-trifluoromethyl-pyridin-2-yl)-piperazin-1-ylmethyl]-1H-pyrazol-3-yl}-benzamide, 2-Cyano-5-{4-[4-(5-trifluoromethyl-pyridin-2-yl)-[1,4]diazepan-1-ylmethyl]-1H-pyrazol-3-yl}-benzamide, 2-Cyano-N-methyl-5-{4-[4-(5-trifluoromethyl-pyridin-2-yl)-[1,4]diazepan-1-ylmethyl]-1H-pyrazol-3-yl}-benzamide, 4-{4-[3-Methyl-4-(5-trifluoromethyl-pyridin-2-yl)-piperazin-1-ylmethyl]-1H-pyrazol-3-yl}-benzonitrile, 4-{4-[3-Methyl-4-(5-trifluoromethyl-pyridin-2-yl)-piperazin-1-ylmethyl]-1H-pyrazol-3-yl 4-benzonitrile, 4-{4-[2-Methyl-4-(5-trifluoromethyl-pyridin-2-yl)-piperazin-1-ylmethyl]-1H-pyrazol-3-yl}-benzonitrile, 4-{4-[5-(5-Trifluoromethyl-pyridin-2-yl)-2,5-diaza-bicyclo[2.2.1]hept-2-ylmethyl]-1H-pyrazol-3-yl}-benzonitrile, 4-{4-[2-Methyl-4-(5-trifluoromethyl-pyridin-2-yl)-piperazin-1-ylmethyl]-1H-pyrazol-3-yl}-benzonitrile, 4-{4-[3,5-Dimethyl-4-(5-trifluoromethyl-pyridin-2-yl)-piperazin-1-ylmethyl]-1H-pyrazol-3-yl}-benzonitrile, 4-{4-[4-(6-Trifluoromethyl-pyridin-3-yl)-piperazin-1-ylmethyl]-1H-pyrazol-3-yl}-benzonitrile, 4-{4-[4-(5-Trifluoromethyl-pyridin-2-yl)-piperazin-1-ylmethyl]-1H-pyrazol-3-yl}-phenol, 1-[3-(4-Bromo-phenyl)-1H-pyrazol-4-ylmethyl]-4-(5-trifluoromethyl-pyridin-2-yl)-piperazine, Ethyl-carbamic acid 4-{4-[4-(5-trifluoromethyl-pyridin-2-yl)-piperazin-1-ylmethyl]-1H-pyrazol-3-yl}-phenyl ester, 1-[3-(4-Imidazol-1-yl-phenyl)-1H-pyrazol-4-ylmethyl]-4-(5-trifluoromethyl-pyridin-2-yl)-piperazine, 2-Methyl-4-{3-[4-(1H-pyrazol-4-yl)-phenyl]-1H-pyrazol-4-ylmethyl}-1-(5-trifluoromethyl-pyridin-2-yl)-piperazine, 4-{4-[3,3-Dimethyl-4-(5-trifluoromethyl-pyridin-2-yl)-piperazin-1-ylmethyl]-1H-pyrazol-3-yl}-benzonitrile, 4-{4-[2,5-Dimethyl-4-(5-trifluoromethyl-pyridin-2-yl)-piperazin-1-ylmethyl]-1H-pyrazol-3-yl}-benzonitrile, Ethyl-carbamic acid 4-{4-[3-methyl-4-(5-trifluoromethyl-pyridin-2-yl)-piperazin-1-ylmethyl]-1H-pyrazol-3-yl}-phenyl ester, 4-{4-[3-Ethyl-4-(5-trifluoromethyl-pyridin-2-yl)-piperazin-1-ylmethyl]-1H-pyrazol-3-yl}-benzonitrile, 1-Ethyl-3-(4-{4-[3-methyl-4-(5-trifluoromethyl-pyridin-2-yl)-piperazin-1-ylmethyl]-1H-pyrazol-3-yl}-phenyl)-urea, Methyl-carbamic acid 4-{4-[4-(5-trifluoromethyl-pyridin-2-yl)-piperazin-1-ylmethyl]-1H-pyrazol-3-yl}-phenyl ester, (4-{4-[3-Methyl-4-(6-trifluoromethyl-pyridin-3-yl)-piperazin-1-ylmethyl]-1H-pyrazol-3-yl}-phenyl)-acetonitrile, 2-(4-{4-[3-Methyl-4-(6-trifluoromethyl-pyridin-3-yl)-piperazin-1-ylmethyl]-1H-pyrazol-3-yl}-phenyl)-acetamide, Dimethyl-(5-{4-[3-methyl-4-(5-trifluoromethyl-pyridin-2-yl)-piperazin-1-ylmethyl]-1H-pyrazol-3-yl}-pyridin-2-yl)-amine, (4-{4-[3-Methyl-4-(6-trifluoromethyl-pyridin-3-yl)-piperazin-1-ylmethyl]-1H-pyrazol-3-yl}-phenyl)-acetic acid, Isopropyl-carbamic acid 4-{4-[3-methyl-4-(5-trifluoromethyl-pyridin-2-yl)-piperazin-1-ylmethyl]-1H-pyrazol-3-yl}-phenyl ester, Phenyl-carbamic acid 4-{4-[3-methyl-4-(5-trifluoromethyl-pyridin-2-yl)-piperazin-1-ylmethyl]-1H-pyrazol-3-yl}-phenyl ester, 5-{4-[3-Methyl-4-(5-trifluoromethyl-pyridin-2-yl)-piperazin-1-ylmethyl]-1H-pyrazol-3-yl}-pyridine-2-carbonitrile, 6-{4-[3-Methyl-4-(5-trifluoromethyl-pyridin-2-yl)-piperazin-1-ylmethyl]-1H-pyrazol-3-yl}-nicotinonitrile, 2-(5-{4-[3-Methyl-4-(5-trifluoromethyl-pyridin-2-yl)-piperazin-1-ylmethyl]-1H-pyrazol-3-yl}-pyridin-2-yl)-acetamide, Carbamic acid 5-{4-[3-methyl-4-(5-trifluoromethyl-pyridin-2-yl)-piperazin-1-ylmethyl]-1H-pyrazol-3-yl}-pyridin-2-yl ester, (S)-methyl 4-((3-(4-cyanophenyl)-1H-pyrazol-4-yl)methyl)-1-(5-(trifluoromethyl)pyridin-2-yl)piperazine-2-carboxylate; (S)-4-((3-(4-cyanophenyl)-1H-pyrazol-4-yl)methyl)-1-(5-(trifluoromethyl)pyridin-2-yl)piperazine-2-carboxylic acid;

(S)-4-(4-((3-(methoxymethyl)-4-(5-(trifluoromethyl)pyridin-2-yl)piperazin-1-yl)methyl)-1H-pyrazol-3-yl)benzonitrile; (R)-2-(4-(4-((3-methyl-4-(5-(trifluoromethyl)pyridin-2-yl)piperazin-1-yl)methyl)-1H-pyrazol-3-yl)phenylamino)ethanol; (R)-5-(4-(4-((3-methyl-4-(5-(trifluoromethyl)pyridin-2-yl)piperazin-1-yl)methyl)-1H-pyrazol-3-yl)phenyl)isoxazole; (R)-4-((3-(4-(1H-pyrrol-1-yl)phenyl)-1H-pyrazol-4-yl)methyl)-2-methyl-1-(5-(trifluoromethyl)pyridin-2-yl)piperazine; (R)-2-methyl-4-((3-(4-(methylsulfonyl)phenyl)-1H-pyrazol-4-yl)methyl)-1-(5-(trifluoromethyl)pyridin-2-yl)piperazine; (R)-N-(4-(4-((3-methyl-4-(5-(trifluoromethyl)pyridin-2-yl)piperazin-1-yl)methyl)-1H-pyrazol-3-yl)phenylsulfonyl)acetamide; (R)-4-(4-((3-methyl-4-(5-(trifluoromethyl)pyridin-2-yl)piperazin-1-yl)methyl)-1H-pyrazol-3-yl)benzoic acid; (R)-4-((3-(4-(1H-tetrazol-5-yl)phenyl)-1H-pyrazol-4-yl)methyl)-2-methyl-1-(5-(trifluoromethyl)pyridin-2-yl)piperazine; (R)-4-((3-(4-((1H-tetrazol-5-yl)methyl)phenyl)-1H-pyrazol-4-yl)methyl)-2-methyl-1-(5-(trifluoromethyl)pyridin-2-yl)piperazine; (R)-2-(5-(4-((3-methyl-4-(5-(trifluoromethyl)pyridin-2-yl)piperazin-1-yl)methyl)-1H-pyrazol-3-yl)pyridin-2-ylamino)ethanol; (R)-2,2′-(5-(4-((3-methyl-4-(5-(trifluoromethyl)pyridin-2-yl)piperazin-1-yl)methyl)-1H-pyrazol-3-yl)pyridin-2-ylazanediyl)diethanol; (S)-4-(4-((3-(trifluoromethyl)-4-(5-(trifluoromethyl)pyridin-2-yl)piperazin-1-yl)methyl)-1H-pyrazol-3-yl)benzonitrile; (R)-4-{4-[3-Trifluoromethyl-4-(5-trifluoromethyl-pyridin-2-yl)-piperazin-1-ylmethyl]-1H-pyrazol-3-yl}-benzonitrile; (S)-4-(4-((2-(trifluoromethyl)-4-(5-(trifluoromethyl)pyridin-2-yl)piperazin-1-yl)methyl)-1H-pyrazol-3-yl)benzonitrile; (R)-4-(4-((2-(trifluoromethyl)-4-(5-(trifluoromethyl)pyridin-2-yl)piperazin-1-yl)methyl)-1H-pyrazol-3-yl)benzonitrile; (R)-4-(4-((2-(fluoromethyl)-4-(5-(trifluoromethyl)pyridin-2-yl)piperazin-1-yl)methyl)-1H-pyrazol-3-yl)benzonitrile; (S)-4-(4-((2-(fluoromethyl)-4-(5-(trifluoromethyl)pyridin-2-yl)piperazin-1-yl)methyl)-1H-pyrazol-3-yl)benzonitrile; 4-(4-((1-(5-(trifluoromethyl)pyridin-2-yl)piperidin-4-yl)methyl)-1H-pyrazol-3-yl)benzonitrile; 4-(4-((4-(5-(trifluoromethyl)pyridin-2-yl)piperidin-1-yl)methyl)-1H-pyrazol-3-yl)benzonitrile; (R)-5-(4-(4-((3-methyl-4-(5-(trifluoromethyl)pyridin-2-yl)piperazin-1-yl)methyl)-1H-pyrazol-3-yl)phenyl)oxazole; (R)-4-((3-(4-(1H-pyrazol-1-yl)phenyl)-1H-pyrazol-4-yl)methyl)-2-methyl-1-(5-(trifluoromethyl)pyridin-2-yl)piperazine; (R)-4-((3-(4-(1H-1,2,4-triazol-1-yl)phenyl)-1H-pyrazol-4-yl)methyl)-2-methyl-1-(5-(trifluoromethyl)pyridin-2-yl)piperazine; (R)-2-(4-(4-((3-methyl-4-(5-(trifluoromethyl)pyridin-2-yl)piperazin-1-yl)methyl)-1H-pyrazol-3-yl)phenylamino)acetic acid; (R)-N-methyl-4-(4-((3-methyl-4-(5-(trifluoromethyl)pyridin-2-yl)piperazin-1-yl)methyl)-1H-pyrazol-3-yl)benzenesulfonamide; (R)-1-(4-(4-((3-methyl-4-(5-(trifluoromethyl)pyridin-2-yl)piperazin-1-yl)methyl)-1H-pyrazol-3-yl)phenyl)-1H-pyrrole-2-carbonitrile; 4-(4-((3-(5-(trifluoromethyl)pyridin-2-yl)-3,8-diazabicyclo[3.2.1]octan-8-yl)methyl)-1H-pyrazol-3-yl)benzonitrile; 4-(4-((8-(5-(trifluoromethyl)pyridin-2-yl)-3,8-diazabicyclo[3.2.1]octan-3-yl)methyl)-1H-pyrazol-3-yl)benzonitrile; (R)-2-methyl-4-((3-(4-(2-methyl-1H-imidazol-1-yl)phenyl)-1H-pyrazol-4-yl)methyl)-1-(5-(trifluoromethyl)pyridin-2-yl)piperazine; (R)-2-methyl-4-((3-(4-(5-methyl-1H-imidazol-1-yl)phenyl)-1H-pyrazol-4-yl)methyl)-1-(5-(trifluoromethyl)pyridin-2-yl)piperazine; (R)-2-methyl-4-((3-(4-(4-methyl-1H-imidazol-1-yl)phenyl)-1H-pyrazol-4-yl)methyl)-1-(5-(trifluoromethyl)pyridin-2-yl)piperazine; (R)-N-(2-(5-(4-((3-methyl-4-(5-(trifluoromethyl)pyridin-2-yl)piperazin-1-yl)methyl)-1H-pyrazol-3-yl)pyridin-2-ylamino)ethyl)acetamide; (R)-N1-(5-(4-((3-methyl-4-(5-(trifluoromethyl)pyridin-2-yl)piperazin-1-yl)methyl)-1H-pyrazol-3-yl)pyridin-2-yl)ethane-1,2-diaamine; (R)-4-(5-(4-((3-methyl-4-(5-(trifluoromethyl)pyridin-2-yl)piperazin-1-yl)methyl)-1H-pyrazol-3-yl)pyridin-2-yl)morpholino; (R)-5-(4-((3-methyl-4-(5-(trifluoromethyl)pyridin-2-yl)piperazin-1-yl)methyl)-1H-pyrazol-3-yl)-N-(2-(piperidin-1-yl)ethyl)pyridin-2-amine; (R)-4-(5-(4-((3-methyl-4-(5-(trifluoromethyl)pyridin-2-yl)piperazin-1-yl)methyl)-1H-pyrazol-3-yl)pyridin-2-yl)piperazin-2-one; (R)-2-hydroxy-4-(4-((3-methyl-4-(5-(trifluoromethyl)pyridin-2-yl)piperazin-1-yl)methyl)-1H-pyrazol-3-yl)benzoic acid; 1-(5-(4-(((R)-3-methyl-4-(5-(trifluoromethyl)pyridin-2-yl)piperazin-1-yl)methyl)-1H-pyrazol-3-yl)pyridin-2-yl)pyrrolidin-3-ol; 4-(4-((7-(5-(trifluoromethyl)pyridin-2-yl)-4,7-diazaspiro[2.5]octan-4-yl)methyl)-1H-pyrazol-3-yl)benzonitrile; 4-(4-((4-(5-(trifluoromethyl)pyridin-2-yl)-4,7-diazaspiro[2.5]octan-7-yl)methyl)-1H-pyrazol-3-yl)benzonitrile; 1-(5-(4-(((R)-3-methyl-4-(5-(trifluoromethyl)pyridin-2-yl)piperazin-1-yl)methyl)-1H-pyrazol-3-yl)pyridin-2-ylamino)propan-2-ol; ((S)-1-(5-(4-(((R)-3-methyl-4-(5-(trifluoromethyl)pyridin-2-yl)piperazin-1-yl)methyl)-1H-pyrazol-3-yl)pyridin-2-yl)pyrrolidin-2-yl)methanol; (R)-1-(5-(4-(((R)-3-methyl-4-(5-(trifluoromethyl)pyridin-2-yl)piperazin-1-yl)methyl)-1H-pyrazol-3-yl)pyridin-2-ylamino)propan-2-ol; (S)-1-(5-(4-(((R)-3-methyl-4-(5-(trifluoromethyl)pyridin-2-yl)piperazin-1-yl)methyl)-1H-pyrazol-3-yl)pyridin-2-ylamino)propan-2-ol; (R)-2-(5-(4-((3-methyl-4-(5-(trifluoromethyl)pyridin-2-yl)piperazin-1-yl)methyl)-1H-pyrazol-3-yl)pyridin-2-yloxy)ethanol; and (R)-1-(5-(4-((3-methyl-4-(5-(trifluoromethyl)pyridin-2-yl)piperazin-1-yl)methyl)-1H-pyrazol-3-yl)pyridin-2-yl)azetidin-3-ol.

Further compounds of the invention are detailed in the Examples and Table I, infra.

### Pharmacology and Utility

Compounds of the invention modulate the activity of IPTKb and, as such, are useful for treating diseases or disorders in which aberrant activity of IPTKb, contributes to the pathology and/or symptomology of diseases.

By inhibiting B cell activation and development, the ITPKb inhibitors of the present invention are useful in various therapeutic applications. Pharmacological inhibition of ITPKb provides a means to inhibit B cell malfunction in pathological settings. For example, B cells play a pathological role in chronic transplant rejection, and the development of autoimmune diseases (e.g. Rheumatoid Arthritis, SLE, lupus, and the like), Psoriasis, Allergy (Asthma, Rhinitis, COPD, Dermatitis) and others, including anaphylaxis and many complement mediated diseases. The ITPKb-inhibiting compounds of the invention can be effective agents to treat these diseases where ITPKb acts to promote pathogenesis.

Other diseases and conditions that are amenable to treatment include diseases associated with or mediated by abnormal B cell proliferation, e.g., B cell lymphoma. They also encompass other antibody-mediated disorders, e.g., allergies, systematic lupus erythematosus (SLE), primary binary cirrhosis (PBC), and idiopathic thrombocytopenic purpura (ITP). In addition to treating these diseases or conditions, ITPKb inhibitors of the present invention are also useful for preventing or modulating the development of such diseases or disorders in a subject (including human and animals such as other mammals) suspected of being, or known to be, prone to such diseases or disorders. The B-cell modulators that can be employed in the therapeutic applications of the invention include the specific ITPKb-inhibitors described in the Examples and tables, infra.

The invention thus provides a method for modulating B lymphocyte development and function in a subject (human or other mammal) for the treatment of autoimmune diseases, the method comprising administering to the subject a compound of formula I or a pharmaceutical composition thereof in an effective amount to modulate the kinase activity or cellular level of ITPKb (such as demonstrated by the in vitro assays described, infra); thereby modulating B lymphocyte differentiation and function in a subject. The compound can down-regulate the cellular level of the ITPKb molecule by inhibiting the kinase activity of ITPKb.

In accordance with the foregoing, the present invention further provides a method for preventing, treating and/or ameliorating the condition of any of the diseases or disorders described above in a subject in need of such treatment, which method comprises administering to said subject a therapeutically effective amount (See, “Administration and Pharmaceutical Compositions”, infra) of a compound of Formula I or a pharmaceutically acceptable salt thereof. Compounds of Formula I can down-regulate the cellular level of the ITPKb molecule by inhibiting the kinase activity of ITPKb such as described by the in vitro assays described, infra. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with other immunomodulatory or anti-inflammatory substances, for example when used in combination with cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, wherein Y is nitrogen and R₆ and R₇ are both hydrogen, can be prepared by proceeding as in the following Reaction Scheme I:

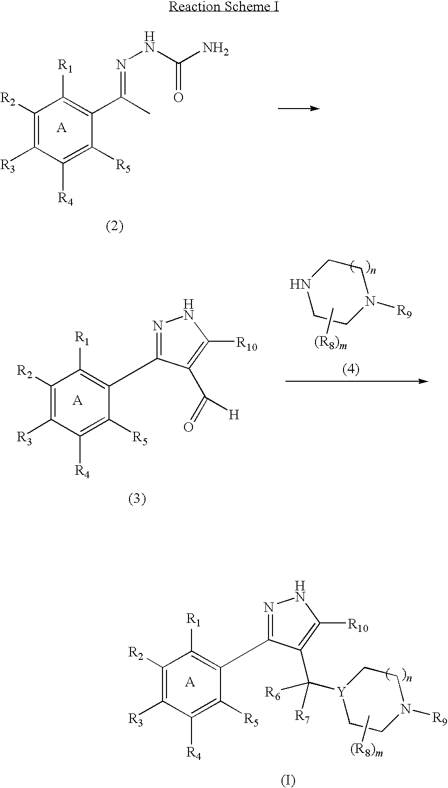

In which n, m, A, R₁, R₂, R₃, R₄, R₅, R₈, R₉ and R₁₀ are as defined in the Summary of the Invention.

A compound of Formula I can be prepared by reacting of a compound of formula 3 with a compound of formula 4 in the presence of a suitable solvent (e.g., DCM) using an appropriate reducing agents (e.g., NaCNBH₃). A compound of formula 3 can be prepared by reacting of a compound formula 2 with the complex of POCl₃ and DMF followed by the addition of a suitable base (e.g., NaOH).

Detailed examples of the synthesis of a compound of Formula I can be found in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, paranitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction scheme I; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### Examples

The present invention is further exemplified, but not limited, by the following examples that illustrate the preparation of compounds of Formula I according to the invention.

### Example 1

### 4-{4-[4-(5-Trifluoromethyl-pyridin-2-yl)-piperazin-1-ylmethyl]-1H-pyrazol-3-yl}-benzonitrile

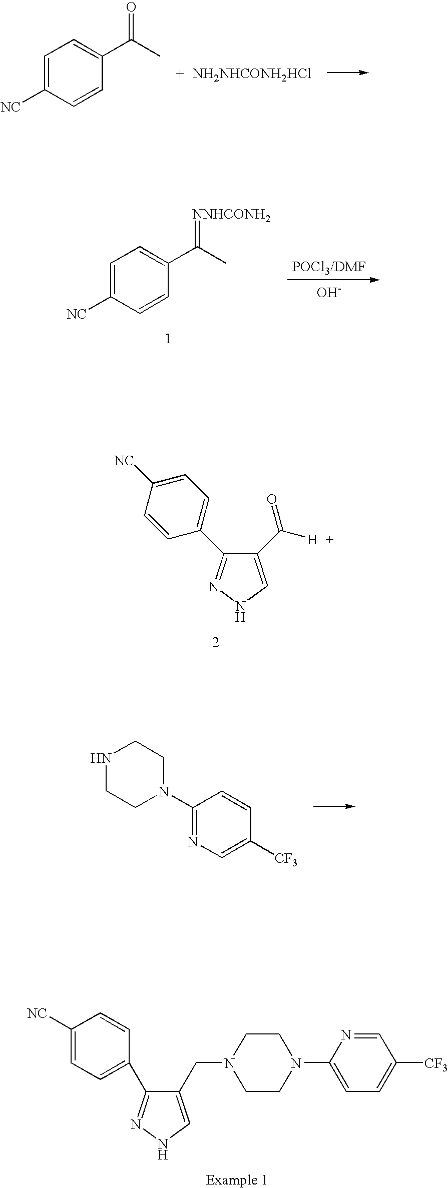

Step 1: To a solution of sodium acetate (51.5 g, 381 mmol) and semicarbazide hydrochloride (23 g, 207 mmol) in water (50 ml) is added a solution of 4-acetyl benzonitrile (25 g, 173 mmol) in ethanol (35 ml). The reaction mixture is heated under reflux for 3 hours. The mixture is cooled to room temperature and crystalline material is formed from the solution which is filtrated and dried in vacuo to give 4-acetyl-benzonitrile semicarbazone as a white solid. ¹H NMR 400 MHz (d-DMSO ) δ 9.60 (s, 1H), 8.06 (d, 2H, J=8.8 Hz), 7.81 (d, 2H, J=8.8 Hz), 6.50 (s, br, 2H), 3.41 (s,br, 1H), 2.20 (s, 3H).

Step 2: The 4-acetyl-benzonitrile (10.1 g, 50 mmol) is added portion wise with stirring to a mixture of phosphorus-dimethylformamide. The latter is prepared by the slow addition of phosphorus oxychloride (10.25 ml, 110 mmol) to dimethylformamide (25 ml, 220 mmol) below 5° C. The reaction mixture is heated at 65° C. for about 4 hours and then is poured into ice after cooling. It is neutralized with sodium hydroxide (20 gram in 80 ml water), and then heated at 55° C. for 10 minutes, cooled and acidified with aqueous concentrated hydrochloric acid. The suspension stands overnight. The precipitated solid is filtered and dried in vacuo to give 3.4 g product as a dark yellow solid. The solution is extracted by EtOAc (50 ml) three times. The combined organic layers are washed with water and brine, dried over MgSO₄. The residue is purified by flash column chromatography (EtOAc/Hexanes=2/5) to 4-(4-formyl-1H-3-yl)-benzonitrile (2.0 g) as a yellow solid. ¹H NMR 400 MHz (d-DMSO) δ 9.93 (s, 1H), 8.70 (s, 1H), 8.12 (d, 2H, J=8 Hz), 7.92 (d, 2H, J=8 Hz).

Step 3: A solution of 4-(4-formyl-1H-3-yl)-benzonitrile (60 mg, 0.3 mmol), 1-[5-(trifluoromethyl)pyrid-2-yl]piperazine (34.7 mg, 0.15 mmol) and glacial acetic acid (25 μL) in methanol (5 mL) is stirred at room temperature for 30 minutes followed by the addition of sodium triacetoxyborohydride (127 mg, 0.6 mmol) in a single portion. The resulting mixture is heated at 40° C. for 1 hour, and then cooled to room temperature. The crude residue is purified by preparative HPLC. The resulting trifluoroacetate salt is neutralized by aqueous concentrated sodium bicarbonate to yield 4-{4-[4-(5-trifluoromethyl-pyridin-2-yl)-piperazin-1-ylmethyl]-1H-pyrazol-3-yl}-benzonitrile as a white solid. ¹H NMR 400 MHz (CDCl₃) δ 8.32 (s, 1H), 7.98 (d, 2H, J=8.4 Hz), 7.65 (d, 2H, J=8.4 Hz), 7.56-7.54 (m, 2H), 6.56 (d, 1H, J=8.8 Hz), 3.59-3.56 (m, 4H), 3.44 (s, 2H), 2.52-2.50 (m, 4H).

### Example 2

### Synthesis of 4-(4-(((R)-4-(5-(trifluoromethyl)pyridin-2-yl)-3-methylpiperazin-1-yl)methyl)-1H-pyrazol-3-yl)phenyl methylcarbamate

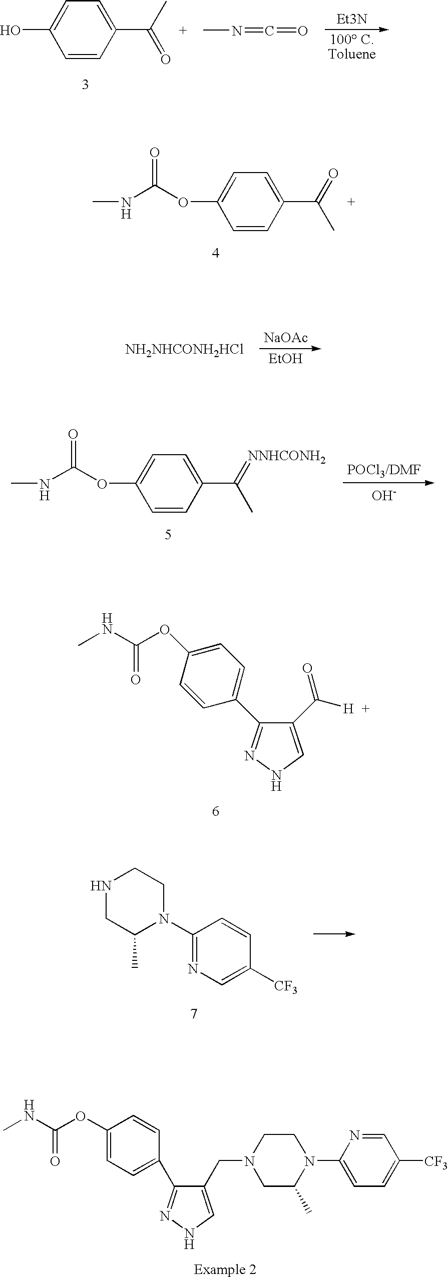

Step 1: 1-(4-hydroxyphenyl) ethanone (3) (544 mg, 4 mmol) and methyl isocynate (500 mg, 8.8 mmol) are mixed in toluene (5 ml) in a sealed tube. To the mixture is added triethylamine (404 mg, 4 mmol) and is heated at 100° C. for 2 hr. The reaction is monitored by LC-MS until (3) disappears. The reaction is quenched by saturated aqueous solution of sodium bicarbonate. The mixture is extracted with EtOAc. (20 ml×5). The combined organic phase is dried over sodium sulfate. After concentration, the crude product is purified by flash chromatography to obtain 4-acetylphenyl methylcarbamate (4) as white solid. 100% (ELSD), m/e: 194 (M+1).

Step 2: 4-acetylphenyl methyl carbamate (4) (750 mg) and semicarbazide hydrochloride (669 mg, 6 mmol) are mixed in ethanol (10 ml). To the mixture is added catalytic amount of acetic acid (0.1 ml). The reaction mixture is heated under reflux for 3 hours. The mixture is cooled to room temperature and crystalline material is formed from the solution which is filtrated and dried in vacuo to give 4-(1-semicarbazidoethyl)phenyl methyl carbamate (5) as a white solid. ¹H NMR 400 MHz (d-Methanol ) δ 7.84-7.81 (m, 2H,), 7.13-7.11 (m, 2H), 2.79 (s, 3H), 2.24 (s, 3H). 100% (ELSD), m/e: 251 (M+1).

Step 3: 4-(1-semicarbazidoethyl)phenyl methyl carbamate (5) (330 mg, 1.32 mmol) is added portion wise with stirring to a mixture of phosphorus-dimethylformamide. The latter is prepared by the slow addition of phosphorus oxychloride (0.41 ml, 4.5 mmol) to dimethylformamide (0.71 ml, 9.0 mmol) below 5° C. The reaction mixture is heated at 65° C. for about 4 hours and then is poured into ice after cooling. It is neutralized with aqueous sodium hydroxide solution (1N), and then heated at 55° C. for 10 minutes, cooled and acidified with aqueous concentrated hydrochloric acid. The solution is extracted by EtOAc (50 ml) three times. The combined organic layers are washed with water and brine, dried over MgSO₄. The residue is purified by flash column chromatography (EtOAc/Hexanes=2/5) to give 4-(4-formyl-1H-pyrazol-3-yl)phenyl methyl carbamate as a yellow solid. 96% (ELSD). m/e: 246 (M+1).

Step 4: A solution of 4-(4-formyl-1H-pyrazol-3-yl)phenyl methyl carbamate (6) (35 mg, 0.142 mmol), (R)-1-(5-(trifluoromethyl)pyridin-2-yl)-2-methylpiperazine (7) (34 mg, 0.14 mmol) and glacial acetic acid (17 μL) in DCM (5 mL) is stirred at room temperature for 30 minutes followed by the addition of sodium triacetoxyborohydride (120 mg, 0.6 mmol) in a single portion. The resulting mixture is heated at 40° C. for 4 hours, and then cooled to room temperature. The crude residue is purified by preparative HPLC using acetic acid as mobile phase to yield 4-(4-(((R)-4-(5-(trifluoromethyl)pyridin-2-yl)-3-methylpiperazin-1-yl)methyl)-1H-pyrazol-3-yl)phenyl methyl carbamate as a white solid. ¹H NMR 400 MHz (d-methanol) δ 8.25 (s, 1H), 7.67 (d, 2H, J=8.0 Hz), 7.63 (m, 1H), 7.12 (d, 2H, J=8.4 Hz), 7.74 (d, 1H J=9.2 Hz), 4.62 (m, 1H), 4.19 (m, 1H), 3.78 (s, 2H), 3.10 (t, 2H, J=12 Hz), 2.97 (m, 1H), 2.70(s, 3H), 2.56(s, 3H, acetate from HPLC), 2.52(m, 1H), 2.32 (m, 1H), 1.14 (d, 3H, J=6.8 Hz). 100 (ELSD), m/e: 475 (M+1).

### Example 3

### 4-[3-(4-Imidazol-1-yl-phenyl)-1H-pyrazol-4-ylmethyl]-2-methyl-1-(5-trifluoromethyl-pyridin-2-yl)-piperazine

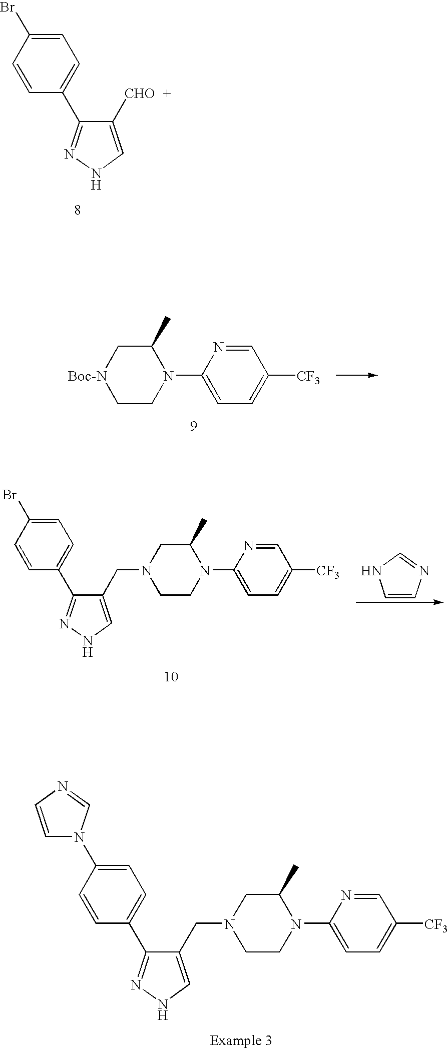

Step 1: To a solution of 3-methyl-4-(5-trifluoromethyl-pyridin-2-yl)-piperazine-1-carboxylic acid tert-butyl ester (9) (200 mg, 0.58 mmol) in dichloromethane (3 ml) is added TFA (1 ml). The reaction mixture is stirred at room temperature for 1 hour. The solvent is removed under vacuum. The residue is dissolved in 1,2-dichloroethane (3 ml). 3-(4-Bromophenyl)-1H-pyrazole-4-carboxaldehyde (132 mg, 0.53 mmol) is added followed by addition of sodium triacetoxyborohydride (223 mg, 1.05 mmol). The reaction mixture is heated at 50° C. overnight. After cooling, the reaction is quenched with saturated NH₄Cl and extracted with AcOEt, then dried (NaSO₄) and concentrated, purified by TLC (Et₃N/MeOH/CH₂Cl₂=3/5/92) to give 4-[3-(4-bromophenyl)-1H-pyrazole-4-ylmethyl]-2-methyl-1-(5-trifluoromethyl-pyridin-2-yl)-piperazine.

Step 2: After standard cycles of evacuation and back-fill with dry and pure argon, an oven-dried Schlenk tube equipped with a magnetic stir bar is charged with Cu₂O (2.1 mg, 0.01 mmol), Salicylaldehyde hydrazone (7.9 mg, 0.06 mmol), imidazole (30 mg, 0.44 mmol), Cs₂CO₃ (171 mg, 0.52 mmol) and 4-[3-(4-bromophenyl)-1H-pyrazole-4-ylmethyl]-2-methyl-1-(5-trifluoromethyl-pyridin-2-yl)-piperazine (140 mg, 0.29 mmol). The tube is evacuated, back-filled with argon. After 1 ml of anhydrous and degassed acetonitrile is added under a stream of argon, the tube is sealed under a positive pressure of argon and heated at 85° C. over weekend. The reaction mixture is allowed to cool to room temperature, diluted with AcOEt and filtered through a plug of Celite. After concentration, the crude residue is purified by preparative HPLC. The resulting TFA salt is neutralized by aqueous NaHCO₃ to give (R)-4-[3-(4-imidazol-1-ylphenyl)-1H-pyrazol-4-ylmethyl]-2-methyl-1-(5-trifluoromethylpyridin-2-yl)-piperazine. ¹H NMR 400 Hz (MeOH-d₄) δ 8.24 (s, 1H), 8.17(s, 1H), 8.02 (d, 2H, J=8.8 Hz), 7.62-7.56(m, 5H), 7.13 (s, 1H), 6.72 (d, 2H, J=9.2 Hz), 4.52(s, 1H), 4.07(d, 1H, J=12.8), 3.41(s, 2H), 3.08(td, 1H, J=12.8, J′=3.2), 2.96(d, 1H, J=11.2), 2.83(d, 1H, J=11.2), 2.21(dd, 1H, J=11.2, J′=4.0), 2.02(td, 1H, J=11.2, J′=3.2) 1.15(d, 3H, J=6.4).

### Example 4

### 4-[3-(6-Chloro-pyridin-3-yl)-1H-pyrazol-4-ylmethyl]-2-(R)-methyl-1-(5-trifluoromethyl-pyridin-2-yl)-piperazine

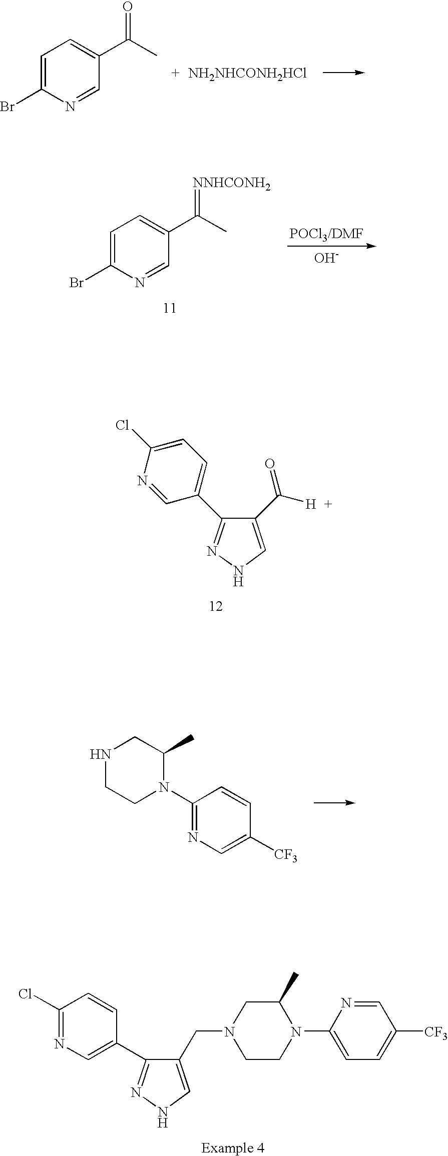

Step 1: To a solution of 5-acetyl-2-bromopyridine (1 g, 5 mmol) in anhydrous ethanol (20 ml) are added semicarbazide hydrochloride (0.61 g, 5.5 mmol) and acetic acid (1 ml). The reaction mixture is heated under reflux for 3 hours. The mixture is cooled to room temperature and the precipitate is filtered and dried in vacuo to yield 5-acetyl-2-bromopyridine semicarbazone. MS, m/e, 257 (M+1).

Step 2: DMF (0.54 ml, 7 mmol) and POCl₃ (0.65 ml, 7 mmol) are separately cooled at 0° C. before POCl₃ is added dropwise to DMF. A solution of 5-acetyl-2-bromopyridine semicarbazone (600 mg, 2.33 mmol) in DMF (5 ml) is added slowly to this reaction mixture. The resulting suspension is then warmed to room temperature and heated at 70° C. for 3 hr. After cooling to room temperature, the mixture is poured to ice and basified with Na₂CO₃. The solution is heated at 60° C. for 10 minutes, cooled, extracted with EtOAc. The combined organic layer is washed with water, dried over Na₂SO₄, filtered and evaporated. The residue is purified by flash chromatography (1:1 EtOAc/Hexanes) to yield 3-(6-Chloro-pyridin-3-yl)-1H-pyrazole-4-carbaldehyde. MS, m/e, 208 (M+1).

Step 3: A solution of 3-(6-Chloro-pyridin-3-yl)-1H-pyrazole-4-carbaldehyde (110 mg, 0.53 mmol), 2-(R)-Methyl-1-(5-trifluoromethyl-pyridin-2-yl)-piperazine (120 mg, 0.49 mmol) and glacial acetic acid (0.2 ml) in anhydrous 1,2-dichloroethane (3 ml) is stirred at 50° C. for 30 minutes followed by the addition of sodium triacetoxyborohydride (210 mg, 1 mmol). The resulting mixture is heated at 50° C. for another 3 hr, and then cooled to room temperature. Ice water is added and the solution is extracted with CH₂Cl₂. The combined organic layers is washed with water, dried over Na₂SO₄, filtered and evaporated. The residue is purified by mass-triggered HPLC. The resulting trifluoroacetate salt is neutralized with aqueous sodium carbonate to yield 4-[3-(6-Chloro-pyridin-3-yl)-1H-pyrazol-4-ylmethyl]-2-(R)-methyl-1-(5-trifluoromethyl-pyridin-2-yl)-piperazine. ¹H NMR 400 MHz (CD3OD) δ 9.0 (s, 1H), 8.45 (d, 1H, J=8.0 Hz), 8.33 (s, 1H), 7.74 (s, 1H), 7.70 (d, 1H, J=8.0 Hz), 7.53 (d, 1H, J=8.0 Hz), 6.81 (d, 1H, J=8 Hz), 4.62 (broad, 1H), 4.20 (broad d, 1H), 3.6-2.8 (m, 5H), 2.4-2.0 (m, 2H), 1.16 (d, 3H, J=7 Hz). MS, m/e, 437 (M+1).

By repeating the procedures described in the above examples, using appropriate starting materials, the following compounds of Formula I, as identified in Table 1, are obtained.

TABLE 1

| Compound | | Physical Data |
|---|---|---|
| Number | Structure | MS (m/z): (M + 1) |
| 5 | 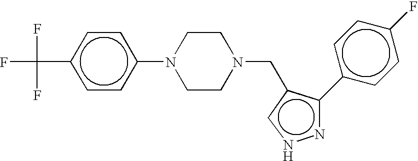 | 405.2 |
| 6 | 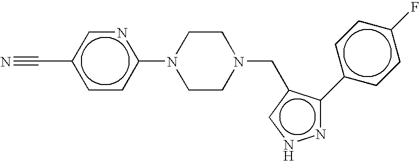 | 363.2 |
| 7 | 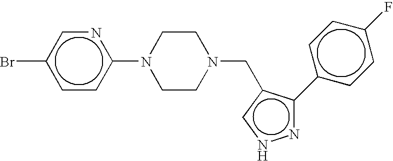 | 416.1 |
| 8 | 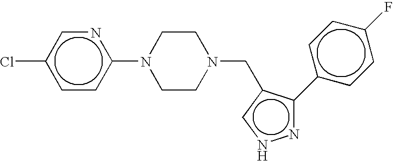 | 372.1 |
| 9 | 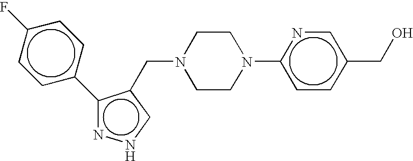 | 368.2 |
| 10 | 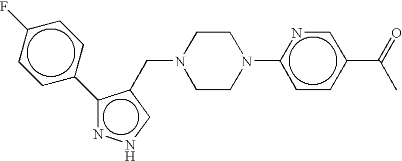 | 380.2 |
| 11 | 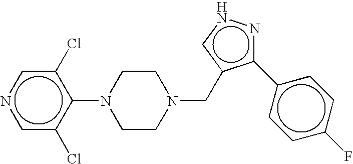 | 406.1 |
| 12 | 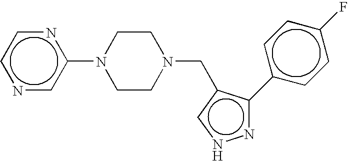 | 339.2 |
| 13 | 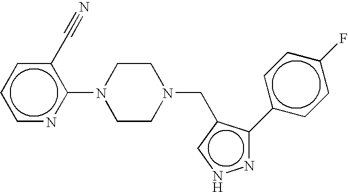 | 363.2 |
| 14 | 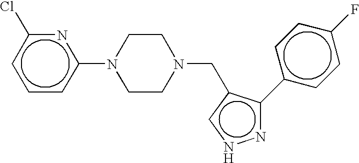 | 372.1 |
| 15 | 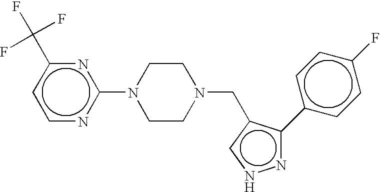 | 407.2 |
| 16 | 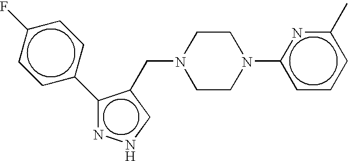 | 352.2 |
| 17 | 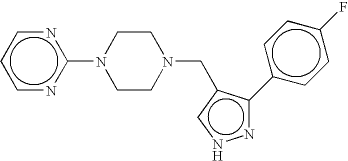 | 339.2 |
| 18 | 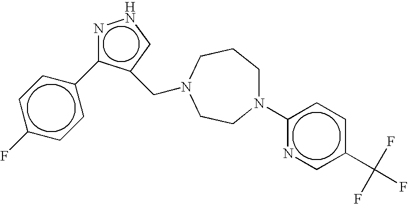 | 420.2 |
| 19 | 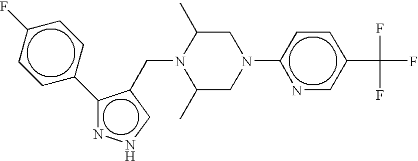 | 434.2 |
| 20 | 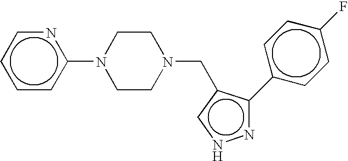 | 338.2 |
| 21 | 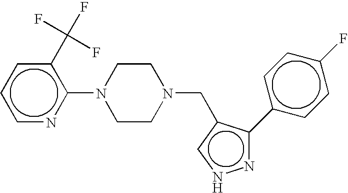 | 406.2 |
| 22 | 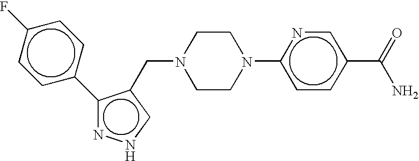 | 381.2 |
| 23 | 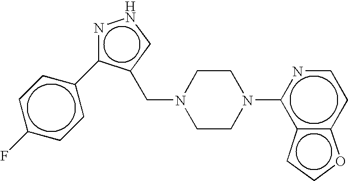 | 378.2 |
| 24 | 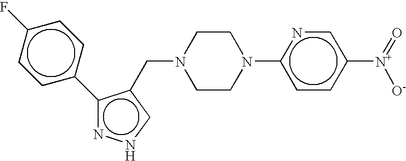 | 383.2 |
| 25 | 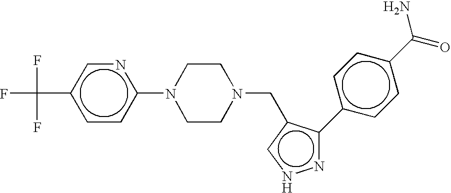 | 431.2 |
| 26 | 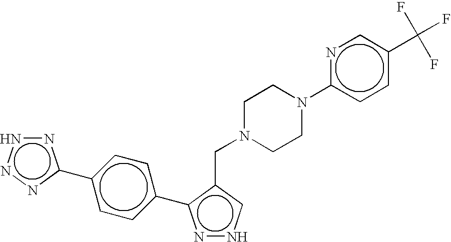 | 456.2 |
| 27 | 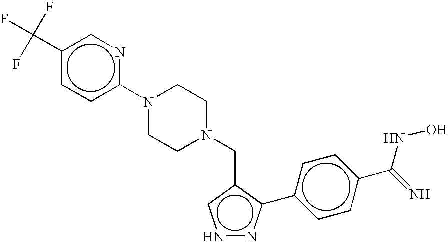 | 446.2 |
| 28 | 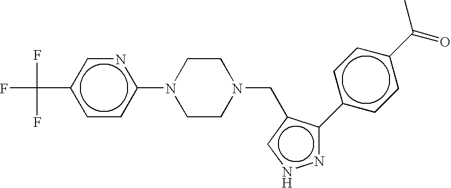 | 430.2 |
| 29 | 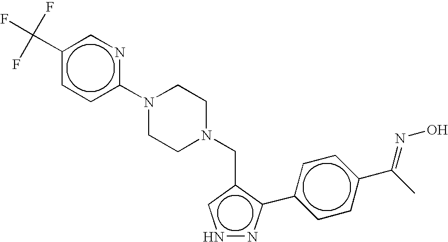 | 445.2 |
| 30 | 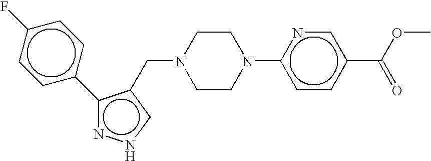 | 396.2 |
| 31 | 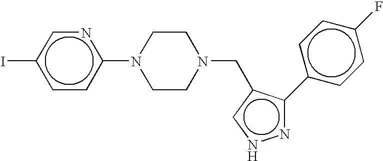 | 464.1 |
| 32 | 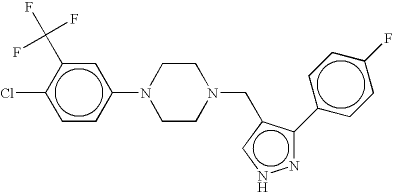 | 439.1 |
| 33 | 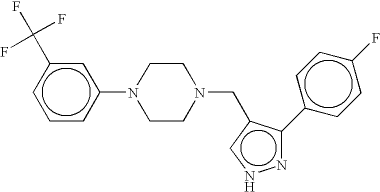 | 405.2 |
| 34 | 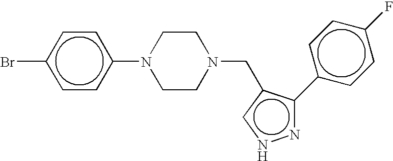 | 415.1 |
| 35 | 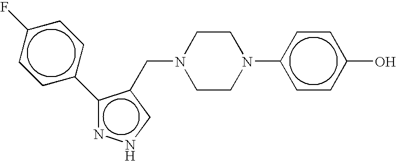 | 353.2 |
| 36 | 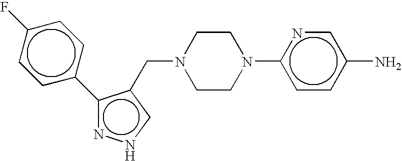 | 353.2 |
| 37 | 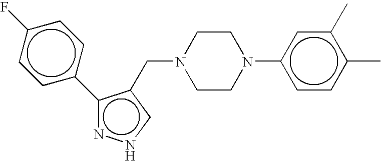 | 365.2 |
| 38 | 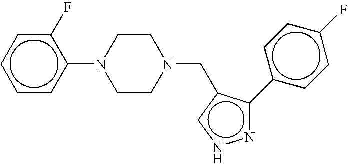 | 355.2 |
| 39 | 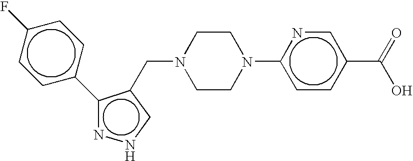 | 382.2 |
| 40 | 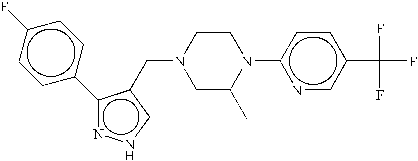 | 420.2 |
| 41 | 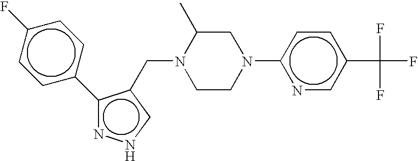 | 420.2 |
| 42 | 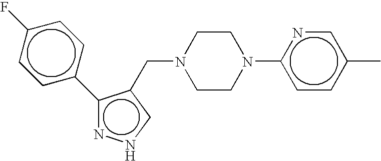 | 352.2 |
| 43 | 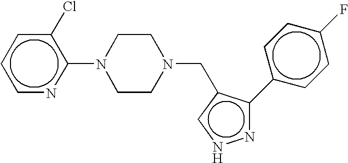 | 372.1 |
| 44 | 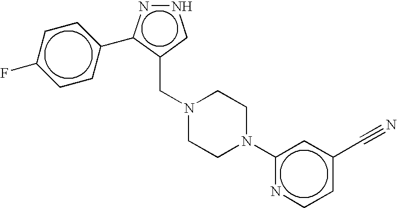 | 363.2 |
| 45 | 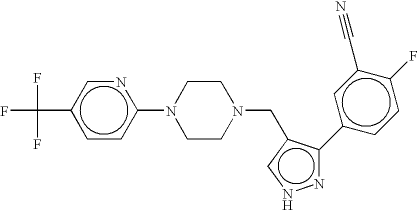 | 431.2 |
| 46 | 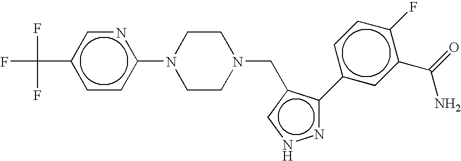 | 449.2 |
| 47 | 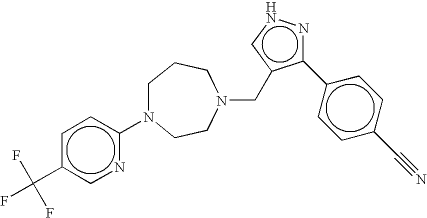 | 427.2 |
| 48 | 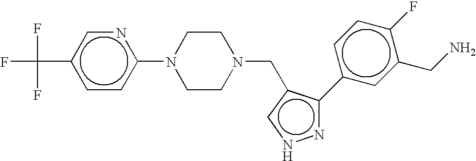 | 435.2 |
| 49 | 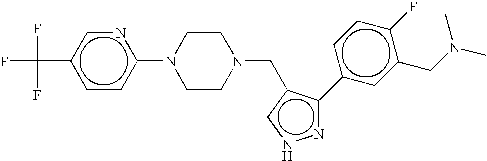 | 463.2 |
| 50 | 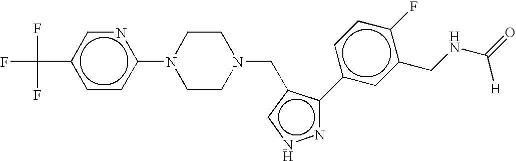 | 463.2 |
| 51 | 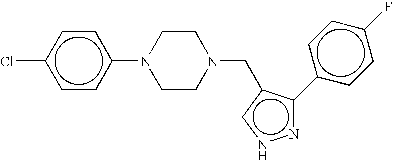 | 371.1 |
| 52 | 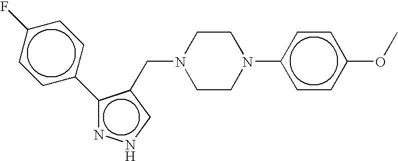 | 367.2 |
| 53 | 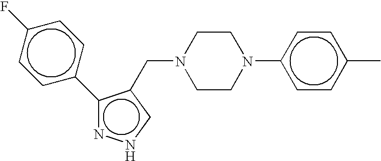 | 351.2 |
| 54 | 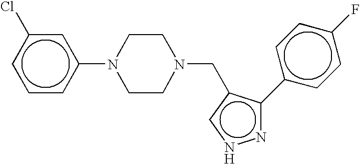 | 371.1 |
| 55 | 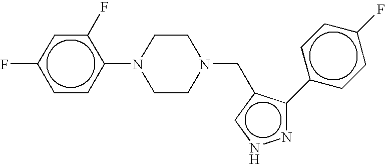 | 373.2 |
| 56 | 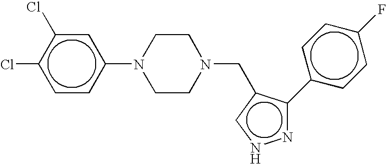 | 405.1 |
| 57 | 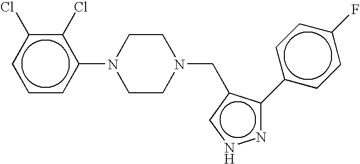 | 405.1 |
| 58 | 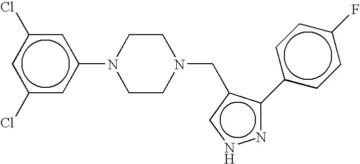 | 405.1 |
| 59 | 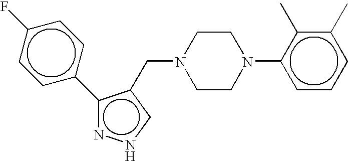 | 365.2 |
| 60 | 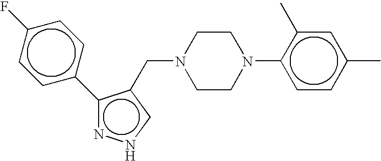 | 365.2 |
| 61 | 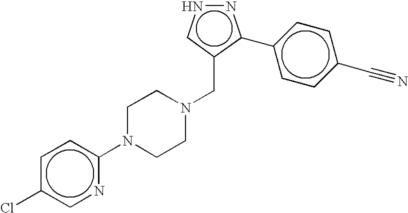 | 379.1 |
| 62 | 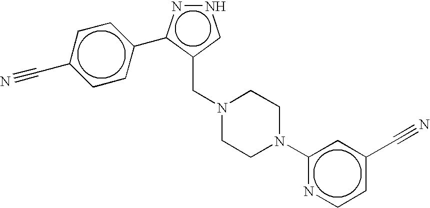 | 370.2 |
| 63 | 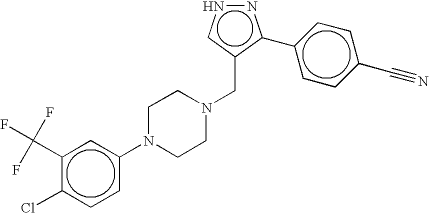 | 446.1 |
| 64 | 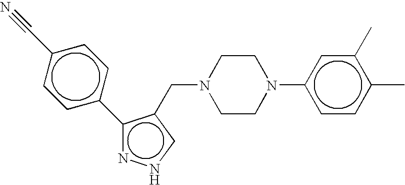 | 372.2 |
| 65 | 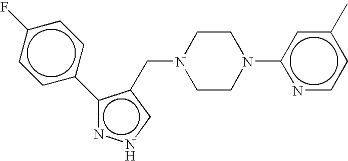 | 352.2 |
| 66 | 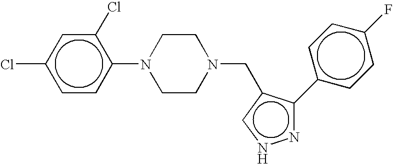 | 405.1 |
| 67 | 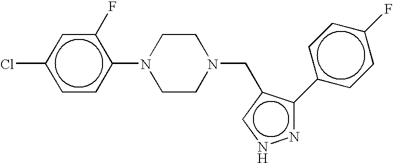 | 389.1 |
| 68 | 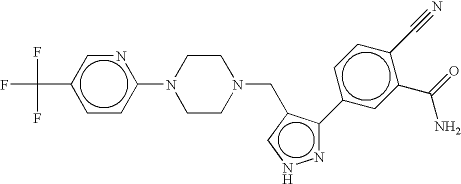 | 456.2 |
| 69 | 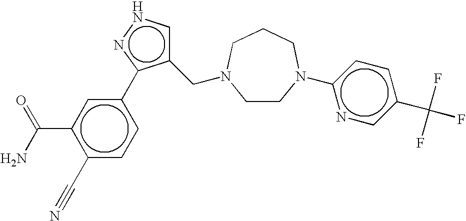 | 470.2 |
| 70 | 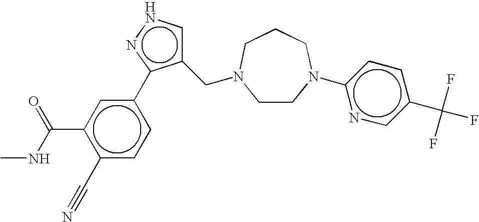 | 484.2 |
| 71 | 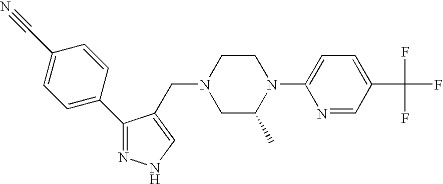 | 427.2 |
| 72 | 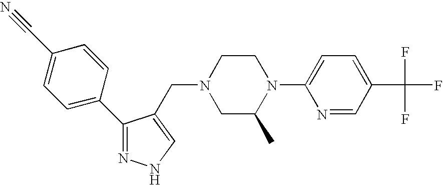 | 427.2 |
| 73 | 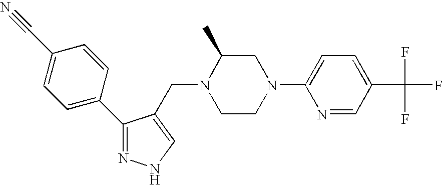 | 427.2 |
| 74 | 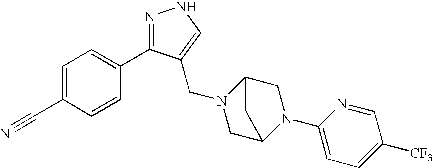 | 425.2 |
| 75 | 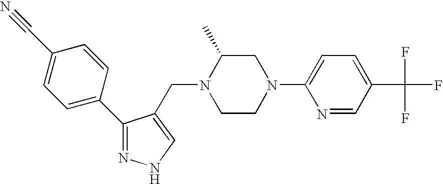 | 427.2 |
| 76 | 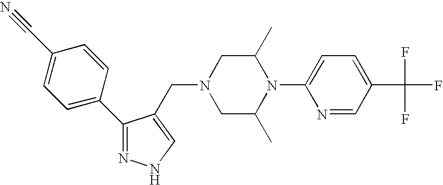 | 441.2 |
| 77 | 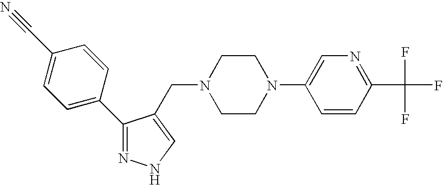 | 413.2 |
| 78 | 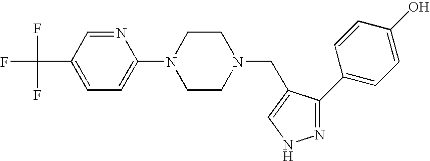 | 404.2 |
| 79 | 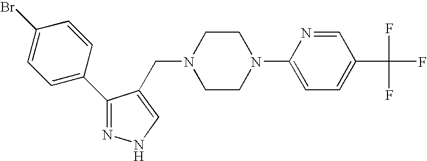 | 466.1 |
| 80 | 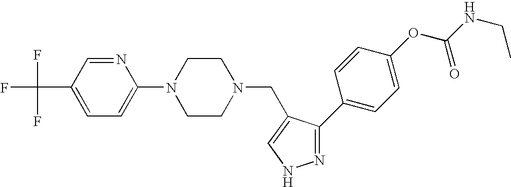 | 475.2 |
| 81 | 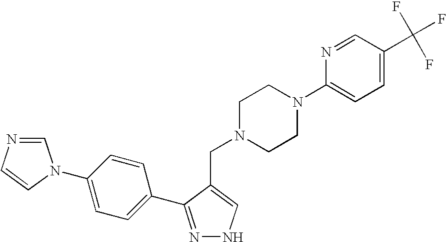 | 454.2 |
| 82 | 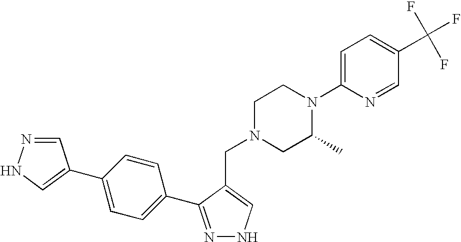 | 468.2 |
| 83 | 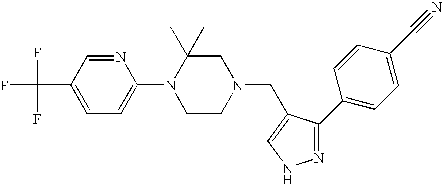 | 441.2 |
| 84 | 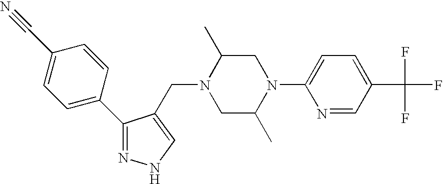 | 441.2 |
| 85 | 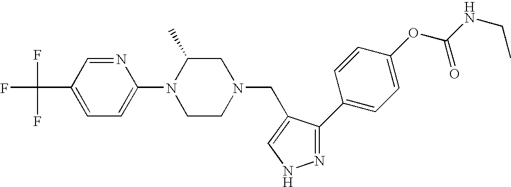 | 489.2 |
| 86 | 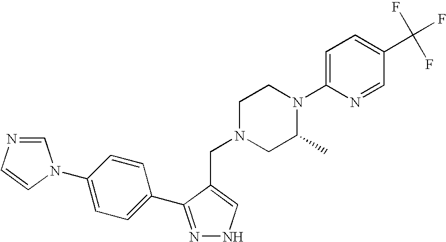 | 468.2 |
| 87 | 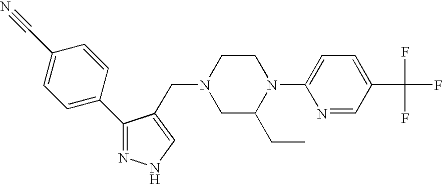 | 441.2 |
| 88 | 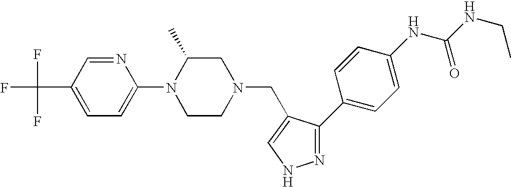 | 488.2 |
| 89 | 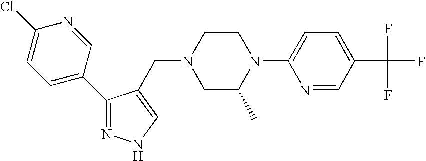 | 437.1 |
| 90 | 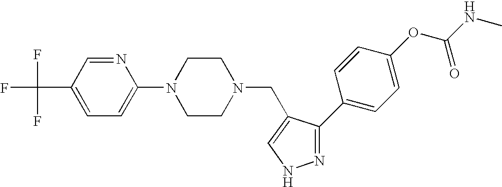 | 461.2 |
| 91 | 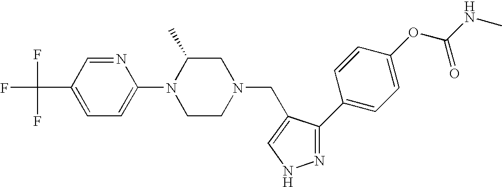 | 475.2 |
| 92 | 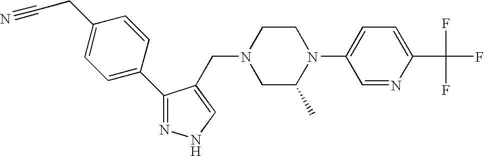 | 441.2 |
| 93 | 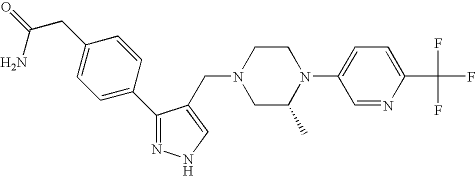 | 459.2 |
| 94 | 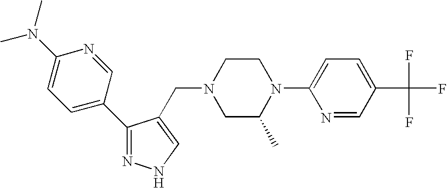 | 446.2 |
| 95 | 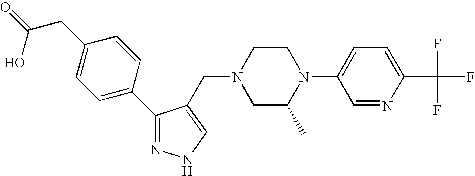 | 460.2 |
| 96 | 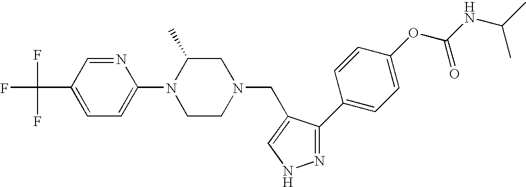 | 503.2 |
| 97 | 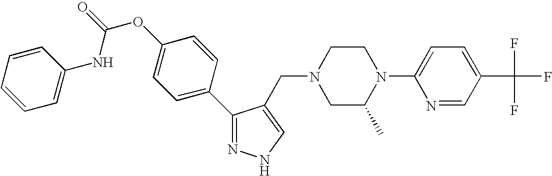 | 537.2 |
| 98 | 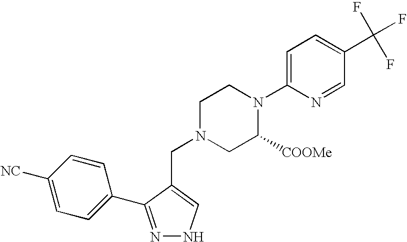 | 471.2 |
| 99 | 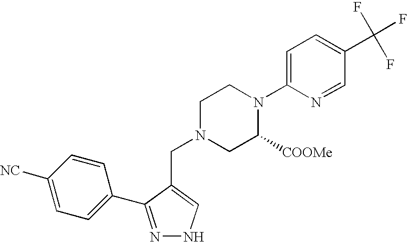 | 457.2 |
| 100 | 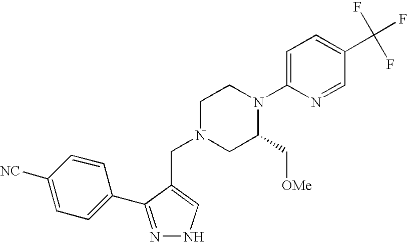 | 457.2 |
| 101 | 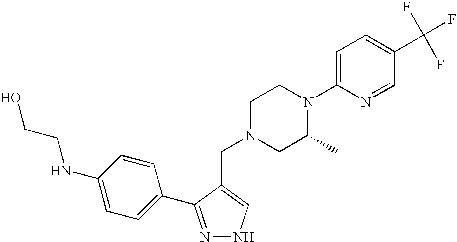 | 461.2 |
| 102 | 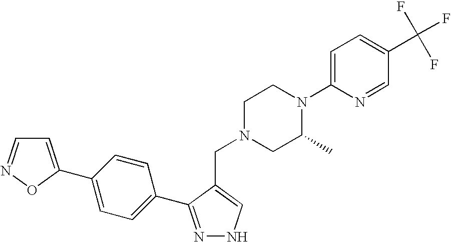 | 469.2 |
| 103 | 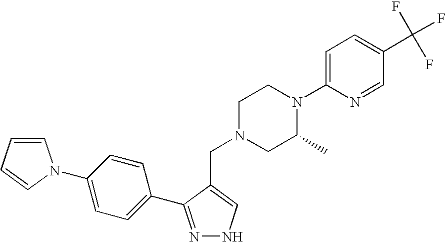 | 467.2 |
| 104 | 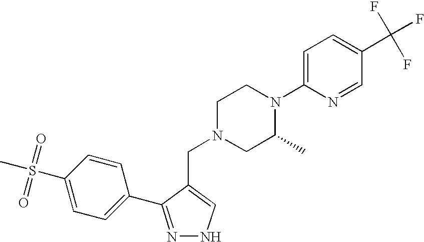 | 480.2 |
| 105 | 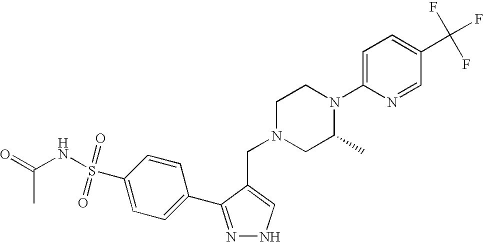 | 423.2 |
| 106 | 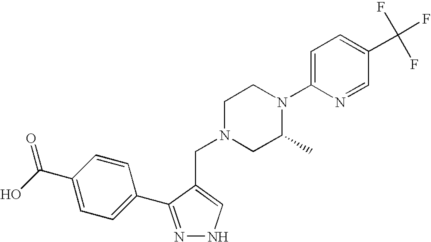 | 446.2 |
| 107 | 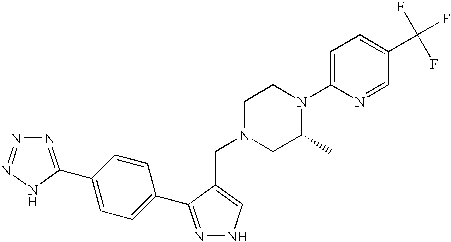 | 470.2 |
| 108 | 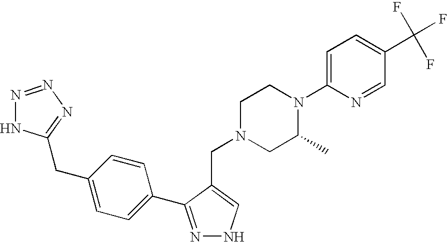 | 484.2 |
| 109 | 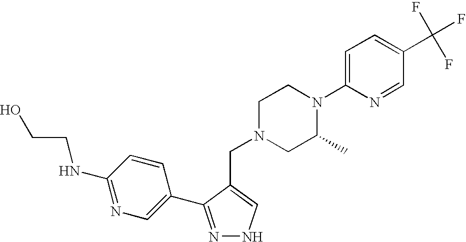 | 462.2 |
| 110 | 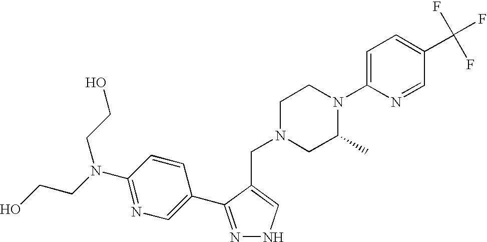 | 506.2 |
| 111 | 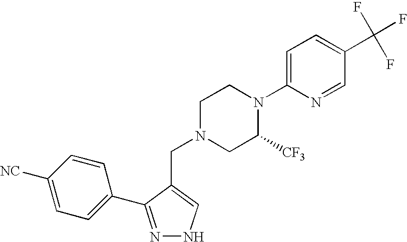 | 481.2 |
| 112 | 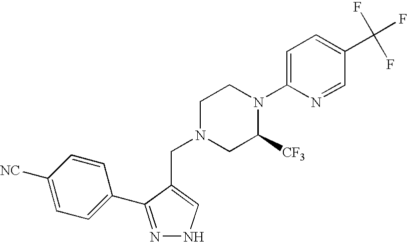 | 481.2 |
| 113 | 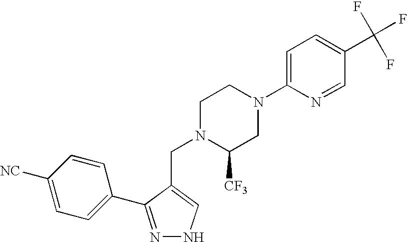 | 481.2 |
| 114 | 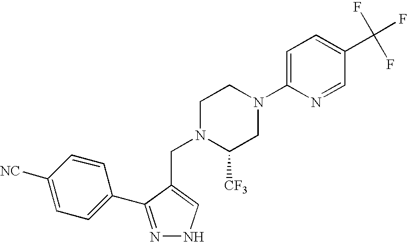 | 481.2 |
| 115 | 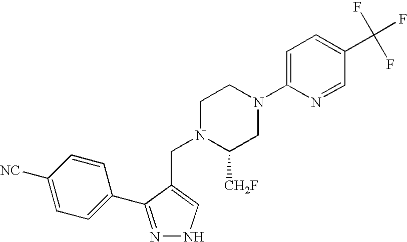 | 445.2 |
| 116 | 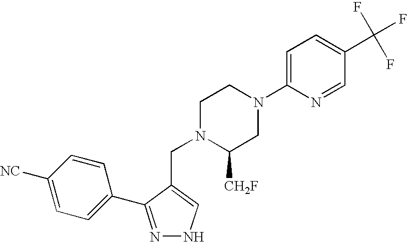 | 445.2 |
| 117 | 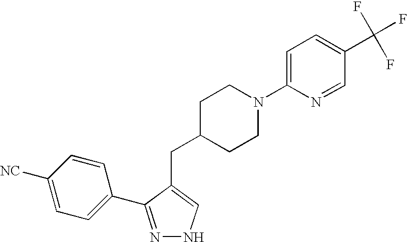 | 412.2 |
| 118 | 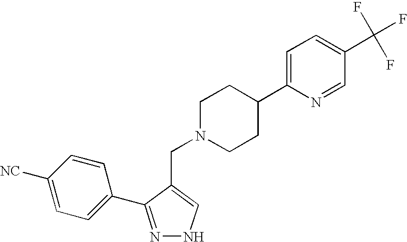 | 412.2 |
| 119 | 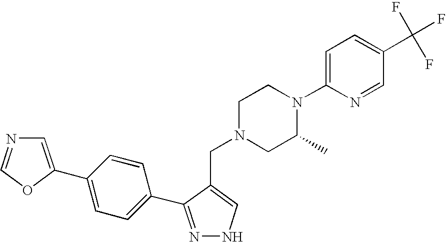 | 469.2 |
| 120 | 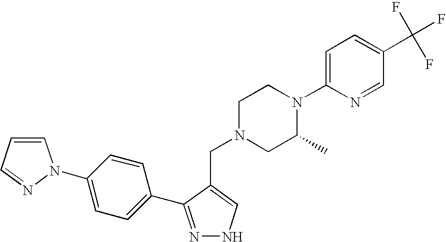 | 468.2 |
| 121 | 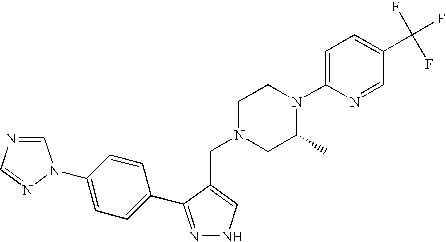 | 469.2 |
| 122 | 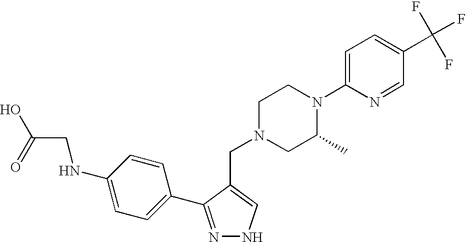 | 475.2 |
| 123 | 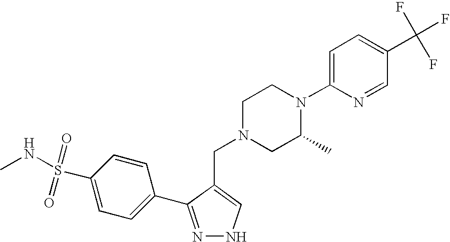 | 495.2 |
| 124 | 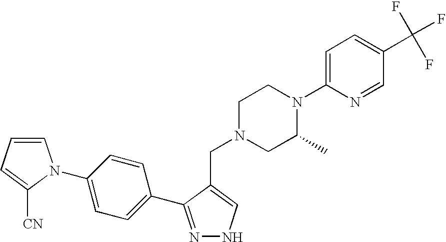 | 492.2 |
| 125 | 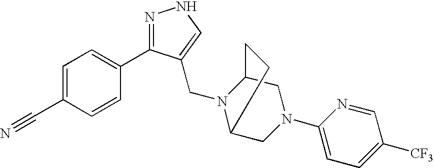 | 439.2 |
| 126 | 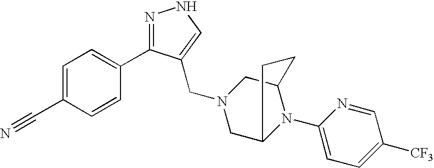 | 439.2 |
| 127 | 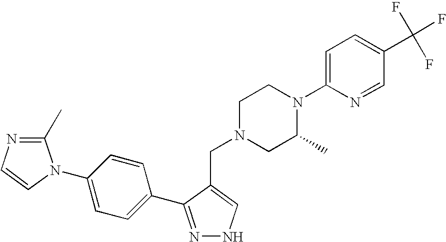 | 482.2 |
| 128 | 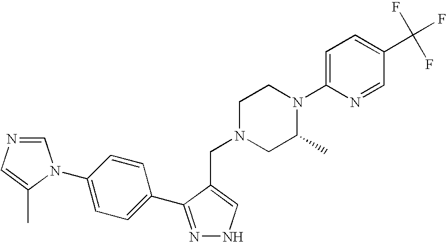 | 482.2 |
| 129 | 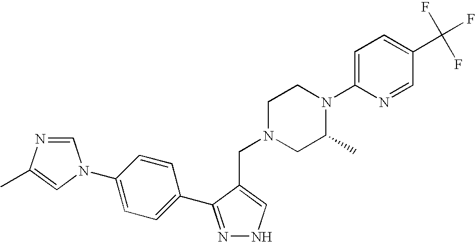 | 482.2 |
| 130 | 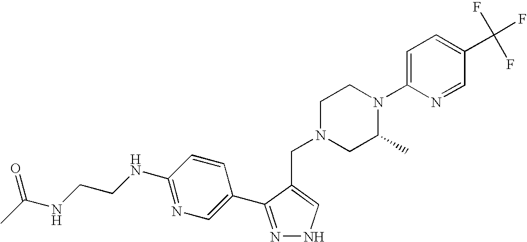 | 503.2 |
| 131 | 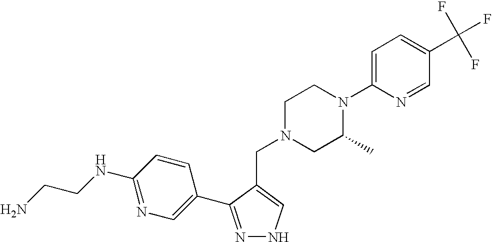 | 461.2 |
| 132 | 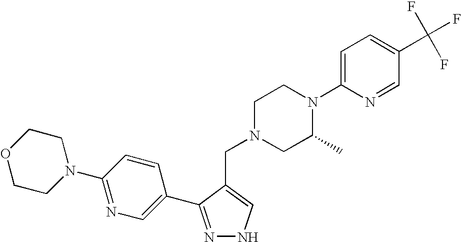 | 488.2 |
| 133 | 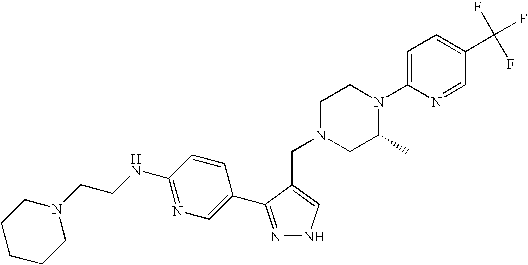 | 529.3 |
| 134 | 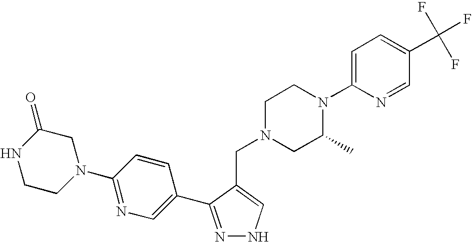 | 501.2 |
| 135 | 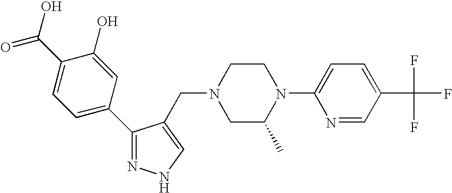 | 462.2 |
| 136 | 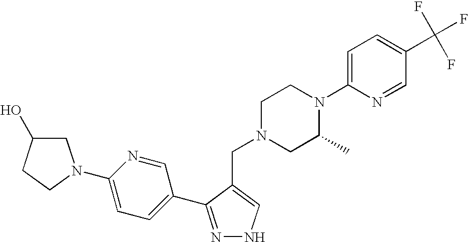 | 488.2 |
| 137 | 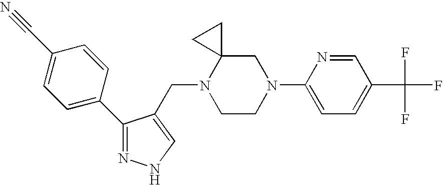 | 439.2 |
| 138 | 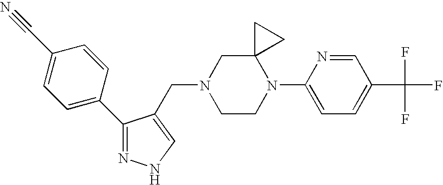 | 439.2 |
| 139 | 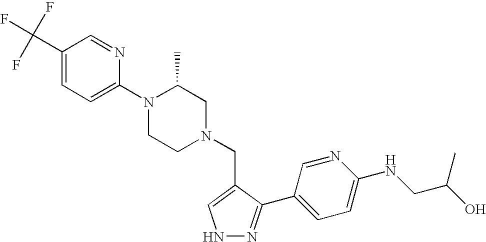 | 476.2 |
| 140 | 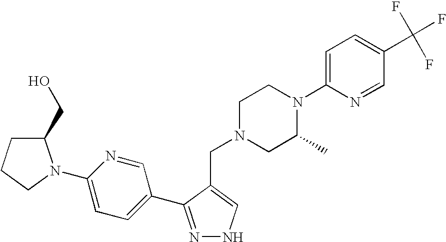 | 502.3 |
| 141 | 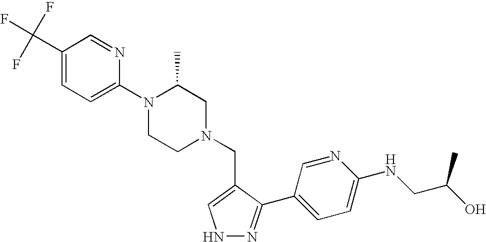 | 476.2 |
| 142 | 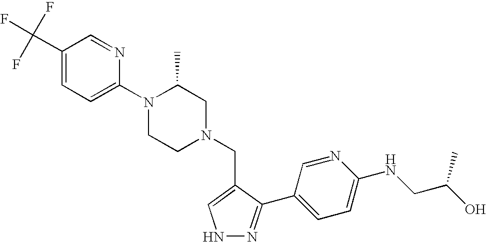 | 476.2 |
| 143 | 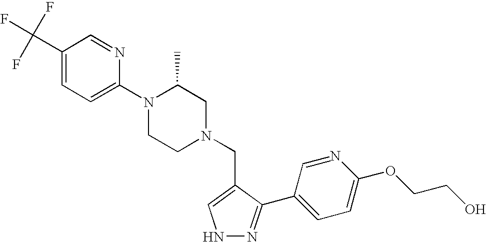 | 463.2 |
| 144 | 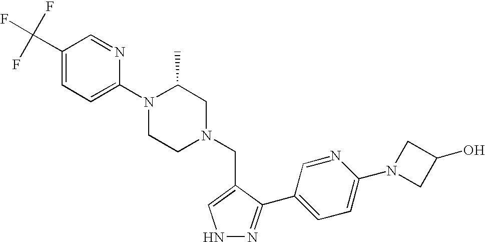 | 474.2 |

### Assays

Compounds of the present invention are assayed to measure their capacity to inhibit ITPKb according to the following assays:

Purification of ITPKb: The DNA sequence encoding murine ITPKb residues 640-942 is amplified from a full-length construct in mammalian expression vector pKDNZ by PCR. The 3′-primer incorporates a stop codon and an overhanging PacI site. The product is digested with PacI before being ligated into the MH4 plasmid which has been prepared by digestion with PmlI and PacI. Cloning into the MH4 plasmid adds the sequence MGSDKIHHHHHH to the N-terminus of the translated region. Mutant enzymes are made by site-directed mutagenesis using the Stratagene Quikchange kit.

ITPKb is expressed in the HK100 strain of Escherichia coli. Typically, a 4 L batch of cells is grown in LB with 0.1 μg/mL ampicillin to 0.5A₆₀₀ at 30 degrees C., before induction with 0.02% L-arabinose for 6 hours. Cells are harvested by centrifugation, and pellets are resuspended in 50 mL of 50 mM Tris (pH 8), 100 mM NaCl, 1 mM TCEP, and 0.1 mg/mL lysozyme, with 1 Complete protease inhibitor tablet (Roche). Cells are disrupted by sonication, and debris is removed by centrifugation for 40 minutes at 35000 g.

Initial purification is performed using three nickel-Sepharose Hi-Trap HP 1 mL columns (Amersham) connected in series. After application of the pellet supernatants, the bound material is washed with 20 mM Tris (pH 8.0), 20 mM imidazole, 10% glycerol (v/v), and 1 mM TCEP before elution with an imidazole gradient up to 200 mM.

Fractions containing ITPKb are identified by SDS-PAGE, and the pure fractions are concentrated and buffer exchanged using centriprep 20 15 kDa columns into 20 mM Tris (pH 8), 200 mM KCl, 5 mM MgCl₂, 0.5 mM DTT, 10% glycerol, 1 μM IP₃, and 20 μM ATP to a final protein concentration of 7 mg/mL.

Biochemical Measurement of ITPKb Activity: ITPKb activity is determined using the Kinase-Glo (Promega) ATP depletion assay. The assay reaction buffer consists of 50 mM Tris (pH 8.0), 100 mM NaCl, 1 mM DTT, 10% glycerol, 5 mM MgCl₂, 1 μM ATP, and 10 μM IP₃ (Alexis Biochemicals). 50 nl of inhibitor is then added to each 40 μL reaction followed by a 10 μL addition of purified ITPKb (final concentration of 60 nM). The reaction mixture is incubated for 60 minutes at room temperature and stopped by the addition of an equal volume of kinase-glo reagent (Promega). Luminescence is measured using a Molecular Devices Acquest instrument.

Compounds of Formula I preferably have an IC₅₀ of less than 500 nM, preferably less than 250 nM, more preferably less than 100 nM at inhibiting the phosphorylation of IP3.

Measuring Intracellular IP3, IP4, and IP5 levels by HPLC: Jurkat cells are obtained from ATCC (clone E6-1) (www.ATCC.org Cat # TIB-152). 10⁷ cells in 1 ml of inositol free RPMI-1640 w/o serum, are pulse labeled at 37° C. for 6 hours with 15 uCi of 3H myo-inositol in inositol. Cells are then diluted to 4 ml of RPMI-1640 with 10% FBS and incubated overnight at 37° C. Cells are then concentrated and resuspended in 1 ml of RPMI-1640 w/10% FBS. 1 μl of inhibitor in DMSO is then added. 50 μg of OKT3 and 10 μg of anti-human CD28 (BD Pharmingen clone CD28.2) is added followed by a 5 minute incubation at 37° C. Cells are then concentrated and the reaction quenched with the resuspension of the cell pellet in 100 μL of PBS w/350 mM HCl. Extracts are then spun to remove proteins and cellular debris. Labeled inositol polyphosphates in the extracts are then resolved by HPLC on a Partisphere SAX column (15 cm×4.6 mm). Samples are eluted as follows with gradients generated by mixing buffer A (10 mM (NH₄)H₂PO₄, pH 3.35, with H₃PO₄) with buffer B (1.7 M (NH₄)H₂PO₄, pH 3.35, with H₃PO₄). 0-12.5 minutes 0-100% Buffer B; 12-5-25 minutes 100% Buffer B; 25-30 minutes 0-100% buffer A; 30-45 minutes 100% buffer A. Radioactivity is detected with an online β-Ram detector from IN/US systems.

Compounds of Formula I preferably have an IC₅₀ of less than 1 μM, more preferably less than 500 nM in inhibiting the conversion of IP3 to IP4.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 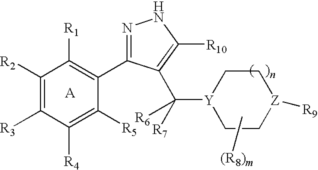
in which:
n is selected from 0, 1, 2 and 3;
m is selected from 0, 1, 2 and 3;
A can have up to 3 groups selected from —CR₁═, —CR₂═, —CR₃═, —CR₄═ and —CR₅═ replaced with N;
R₁, R₂, R₃, R₄ and R₅ are independently selected from hydrogen, hydroxy, halo, cyano, C₁₋₆alkyl, halo-substituted-C₁₋₆alkyl, hydroxy-substituted-C₁₋₆alkyl, cyano-substituted-C₁₋₆alkyl, C₃₋₈heterocycloalkyl-C₀₋₄alkyl, C₁₋₁₀heteroaryl-C₀₋₄alkyl, —XSO₂R₁₁, —XSO₂NR₁₁R₁₂, —XSO₂NR₁₁C(O)R₁₂, —XC(NR₁₁)NR₁₁OR₁₂, —XCR₁₁═NOR₁₂, —XC(O)R₁₁, —XC(O)OR₁₁, —XNR₁₁R₁₂, —XC(O)NR₁₁R₁₂, —XOC(O)NR₁₁R₁₂, —XNR₁₁C(O)NR₁₁R₁₂, —XNR₁₁XOR₁₂, —XN(XOR₁₂)₂, —XNR₁₁XC(O)OR₁₂, —XNR₁₁XNR₁₁C(O)R₁₂, —XNR₁₁XNR₁₁R₁₂, —XNR₁₁C(O)R₁₂; wherein each X is independently selected from a bond and C₁₋₄alkylene; each R₁₁ is selected from hydrogen and C₁₋₆alkyl; and R₁₂ is selected from hydrogen, C₁₋₆alkyl and C₆₋₁₀aryl; or R₁₁ and R₁₂ together with the nitrogen to which R₁₁ and R₁₂ are attached form a C₃₋₈heterocycloalkyl; wherein said heteroaryl or heterocycloalkyl of R₁, R₂, R₃, R₄ or R₅ is optionally substituted with 1 to 3 radicals independently selected from halo, hydroxy, cyano, C₁₋₆alkyl, halo-substituted-C₁₋₆alkyl, hydroxy-substituted-C₁₋₆alkyl, cyano-substituted-C₁₋₆alkyl and carboxy;
R₆ and R₇ are independently selected from hydrogen and C₁₋₃alkyl; or R₆ and R₇, together with the carbon to which they are both attached, forms C₃₋₇cycloalkyl;
R₈ is selected from C₁₋₆alkyl, halo-substituted-C₁₋₃alkyl, C₁₋₆ alkoxy, —CH₂OR₈ₐ, —COOR₈ₐ and C₂₋₆alkenyl; or two R₈ groups attached to different carbon atoms can combine to form an alkyl bridge; or two R₈ groups attached to the same carbon can form a C₃₋₈cycloalkyl group or a carbonyl group; wherein R₈ₐ is selected from hydrogen and C₁₋₆alkyl;
R₉ is selected from C₆₋₁₀aryl and C₁₋₁₀heteroaryl; wherein said aryl or heteroaryl of R₉ is optionally substituted with 1 to 3 radicals independently selected from halo, cyano, hydroxy, C₁₋₃alkyl, halo-substituted-C₁₋₃alkyl, cyano-substituted-C₁₋₃alkyl, hydroxy-substituted-C₁₋₃alkyl, —C(O)R₁₃, —C(O)NR₁₃R₁₄; wherein each R₁₃ and R₁₄ are independently selected from hydrogen and C₁₋₆alkyl;
R₁₀ is selected from hydrogen, C₁₋₆alkyl, —NR₁₅R₁₆, —NR₁₅C(O)R₁₆ and —C(O)NR₁₅R₁₆; wherein each R₁₅ and R₁₆ are independently selected from hydrogen, C₁₋₆alkyl, C₆₋₁₀aryl, C₁₋₁₀heteroaryl, C₃₋₁₂cycloalkyl and C₃₋₈heterocycloalkyl; wherein said aryl, heteroaryl, cycloalkyl and heterocycloalkyl can be optionally substituted with 1 to 3 radicals independently selected from halo, hydroxy, cyano, C₁₋₆alkyl, halo-substituted-C₁₋₆alkyl, C₁₋₆alkoxy and halo-substituted-C₁₋₆alkoxy;
Y and Z are independently selected from CR₂₀ and N; wherein R₂₀ is selected from hydrogen and C₁₋₄alkyl; and the pharmaceutically acceptable salts thereof; with the proviso that compounds of Formula I do not include compounds of Formula II.

2. The compound of claim 1 in which:
n is selected from 1 and 2;
m is selected from 0, 1 and 2;
A can have up to 3 groups selected from —CR₁═, —CR₂═, —CR₃═, —CR₄═ and —CR₅═ replaced with N;
R₂, R₃ and R₄ are independently selected from hydrogen, hydroxy, halo, cyano, C₁₋₆alkyl, halo-substituted-C₁₋₆alkyl, hydroxy-substituted-C₁₋₆alkyl, cyano-substituted-C₁₋₆alkyl, C₃₋₈heterocycloalkyl-C₀₋₄alkyl, C₁₋₁₀heteroaryl-C₀₋₄alkyl, —XSO₂R₁₁, —XSO₂NR₁₁R₁₂, —XSO₂NR₁₁C(O)R₁₂, —XC(NR₁₁)NR₁₁OR₁₂, —XCR₁₁═NOR₁₂, —XC(O)R₁₁, —XC(O)OR₁₁, —XNR₁₁R₁₂, —XC(O)NR₁₁R₁₂, —XOC(O)NR₁₁R₁₂, —XNR₁₁C(O)NR₁₁R₁₂, —XNR₁₁XOR₁₂, —XN(XOR₁₂)₂, —XNR₁₁XC(O)OR₁₂, —XNR₁₁C(O)R₁₂; wherein each X is independently selected from a bond and C₁₋₄alkylene; each R₁₁ is selected from hydrogen and C₁₋₆alkyl; and R₁₂ is selected from hydrogen, C₁₋₆alkyl and C₆₋₁₀aryl; wherein said heteroaryl or heterocycloalkyl of R₁, R₂, R₃, R₄ or R₅ is optionally substituted with 1 to 3 radicals independently selected from halo, hydroxy, cyano, C₁₋₆alkyl, halo-substituted-C₁₋₆alkyl, hydroxy-substituted-C₁₋₆alkyl, cyano-substituted-C₁₋₆alkyl and carboxy;
R₁ and R₅ are hydrogen;
R₆ and R₇ are hydrogen;
R₈ is selected from C₁₋₂alkyl, halo-substituted-C₁₋₃alkyl, C₁₋₆ alkoxy, —CH₂OR₈ₐ, —COOR₈ₐ and C₂₋₆alkenyl; or two R₈ groups attached to different carbon atoms can combine to form an alkyl bridge; or two R₈ groups attached to the same carbon can form a C₃₋₈cycloalkyl group or a carbonyl group; wherein R₈ₐ is selected from hydrogen and C₁₋₆alkyl;
R₉ is selected from C₆₋₁₀aryl and C₁₋₁₀heteroaryl; wherein said aryl or heteroaryl of R₉ is optionally substituted with 1 to 3 radicals independently selected from halo, cyano, hydroxy, C₁₋₃alkyl, halo-substituted-C₁₋₃alkyl, cyano-substituted-C₁₋₃alkyl, hydroxy-substituted-C₁₋₃alkyl, —C(O)R₁₃, —C(O)NR₁₃R₁₄; wherein each R₁₃ and R₁₄ are independently selected from hydrogen and C₁₋₆alkyl; and
R₁₀ is hydrogen.

3. The compound of claim 2 in which Y is N; and A can has a group selected from —CR₁═, —CR₂═, —CR₃═, —CR₄═ and —CR₅═ replaced with N.

4. The compound of claim 3 in which R₂, R₃ and R₄ are independently selected from hydrogen, hydroxy, cyano, cyano-methyl, fluoro, chloro, bromo, iodo, amino-carbonyl, amino-carbonyl-methyl, tetrazolyl, amidino, methyl-carbonyl, 1-(hydroxy-imino)ethyl, amino-methyl, dimethyl-amino-methyl, N-ethylformamide, methyl-amino-carbonyl, dimethyl-amino, carboxy-methyl, methyl-amino-carboxy, ethyl-amino-carboxy, imidazolyl, pyrazolyl, 3-ethylureido, isopropyl-amino-carboxy, phenyl-amino-carboxy, hydroxy-carbonyl-methyl-amino, 2-hydroxy-ethoxy, 2-hydroxypropylamino, amino-carboxy, hydroxy-ethyl-amino, pyrrolidinyl substituted with carboxy, isoxazolyl, 2-hydroxy-methyl-pyrrolidin-1-yl, 3-hydroxypyrrolidin-1-yl, 3-hydroxyazetidin-1-yl, pyrrolyl optionally substituted with cyano, methyl-amino-sulfonyl, methyl-sulfonyl, methyl-carbonyl-amino-sulfonyl, carboxy, tetrazolyl, tetrazolyl-methyl, dihydroxyethyl-amino, oxazolyl, imidazolyl optionally substituted with methyl, pyrazolyl and 1,2,4-trazolyl.

5. The compound of claim 4 in which R₈ is selected from methyl, ethyl, methoxy-carbonyl, carboxy, trifluoromethyl and fluoromethyl; or two R₈ groups can combine to form an ethyl or propyl bridge; or two R₈ groups attached to the same carbon can form cyclopropyl.

6. The compound of claim 5 in which R₉ is selected from phenyl, pyridinyl, pyrazinyl, pyrimidinyl and furo[3,2-c]pyridin-4-yl; wherein said phenyl, pyridinyl, pyrazinyl, pyrimidinyl or furo[3,2-c]pyridin-4-yl is optionally substituted with 1 to 3 radicals independently selected from trifluoromethyl, cyano, bromo, chloro, hydroxy-methyl, methyl-carbonyl, methyl, amino-carbonyl, nitro, iodo, fluoro, methoxy-carbonyl, hydroxy, amino, carboxy and methoxy.

7. The compound of claim 1 selected from 4-{4-[4-(5-Trifluoromethyl-pyridin-2-yl)-piperazin-1-ylmethyl]-1H-pyrazol-3-yl}-benzonitrile, Methyl-carbamic acid 4-{4-[3-methyl-4-(5-trifluoromethyl-pyridin-2-yl)-piperazin-1-ylmethyl]-1H-pyrazol-3-yl}-phenyl ester, 4-[3-(4-Imidazol-1-yl-phenyl)-1H-pyrazol-4-ylmethyl]-2-methyl-1-(5-trifluoromethyl-pyridin-2-yl)-piperazine, 4-[3-(6-Chloro-pyridin-3-yl)-1H-pyrazol-4-ylmethyl]-2-methyl-1-(5-trifluoromethyl-pyridin-2-yl)-piperazine, 1-[3-(4-Fluoro-phenyl)-1H-pyrazol-4-ylmethyl]-4-(4-trifluoromethyl-phenyl)-piperazine, 6-{4-[3-(4-Fluoro-phenyl)-1H-pyrazol-4-ylmethyl-]-piperazin-1-yl}-nicotinonitrile, 1-(5-Bromo-pyridin-2-yl)-4-[3-(4-fluoro-phenyl)-1H-pyrazol-4-ylmethyl]-piperazine, 1-(5-Chloro-pyridin-2-yl)-4-[3-(4-fluoro-phenyl)-1H-pyrazol-4-ylmethyl]-piperazine, (6-{4-[3-(4-Fluoro-phenyl)-1H-pyrazol-4-ylmethyl]-piperazin-1-yl}-pyridin-3-yl)-methanol, 1-(6-{4-[3-(4-Fluoro-phenyl)-1H-pyrazol-4-ylmethyl]-piperazin-1-yl}-pyridin-3-yl)-ethanone, 1-(3,5-Dichloro-pyridin-4-yl)-4-[3-(4-fluoro-phenyl)-1H-pyrazol-4-ylmethyl]-piperazine, 4-[3-(4-Fluoro-phenyl)-1H-pyrazol-4-ylmethyl]-3,4,5,6-tetrahydro-2H-[1,2′]bipyrazinyl, 2-{4-[3-(4-Fluoro-phenyl)-1H-pyrazol-4-ylmethyl]-piperazin-1-yl}-nicotinonitrile, 1-(6-Chloro-pyridin-2-yl)-4-[3-(4-fluoro-phenyl)-1H-pyrazol-4-ylmethyl]-piperazine, 2-{4-[3-(4-Fluoro-phenyl)-1H-pyrazol-4-ylmethyl]-piperazin-1-yl}-4-trifluoromethyl-pyrimidine, 1-[3-(4-Fluoro-phenyl)-1H-pyrazol-4-ylmethyl]-4-(6-methyl-pyridin-2-yl)-piperazine, 2-{4-[3-(4-Fluoro-phenyl)-1H-pyrazol-4-ylmethyl]-piperazin-1-yl}-pyrimidine, 1-[3-(4-Fluoro-phenyl)-1H-pyrazol-4-ylmethyl]-4-(5-trifluoromethyl-pyridin-2-yl)-[1,4]diazepane, 1-[3-(4-Fluoro-phenyl)-1H-pyrazol-4-ylmethyl]-2,6-dimethyl-4-(5-trifluoromethyl-pyridin-2-yl)-piperazine, 1-[3-(4-Fluoro-phenyl)-1H-pyrazol-4-ylmethyl]-4-pyridin-2-yl-piperazine, 1-[3-(4-Fluoro-phenyl)-1H-pyrazol-4-ylmethyl]-4-(3-trifluoromethyl-pyridin-2-yl)-piperazine, 6-{4-[3-(4-Fluoro-phenyl)-1H-pyrazol-4-ylmethyl]-piperazin-1-yl}-nicotinamide, 4-{4-[3-(4-Fluoro-phenyl)-1H-pyrazol-4-ylmethyl]-piperazin-1-yl}-furo[3,2-c]pyridine, 1-[3-(4-Fluoro-phenyl)-1H-pyrazol-4-ylmethyl]-4-(5-nitro-pyridin-2-yl)-piperazine, 4-{4-[4-(5-Trifluoromethyl-pyridin-2-yl)-piperazin-1-ylmethyl]-1H-pyrazol-3-yl}-benzamide, 1-{3-[4-(1H-Tetrazol-5-yl)-phenyl]-1H-pyrazol-4-ylmethyl}-4-(5-trifluoromethyl-pyridin-2-yl)-piperazine, N-Hydroxy-4-{4-[4-(5-trifluoromethyl-pyridin-2-yl)-piperazin-1-ylmethyl]-1H-pyrazol-3-yl}-benzamidine, 1-(4-{4-[4-(5-Trifluoromethyl-pyridin-2-yl)-piperazin-1-ylmethyl]-1H-pyrazol-3-yl}-phenyl)-ethanone, 1-(4-{4-[4-(5-Trifluoromethyl-pyridin-2-yl)-piperazin-1-ylmethyl]-1H-pyrazol-3-yl}-phenyl)-ethanone oxime, 4-{4-[4-(5-Trifluoromethyl-pyridin-2-yl)-piperazin-1-ylmethyl]-1H-pyrazol-3-yl}-benzoic acid methyl ester, 1-[3-(4-Fluoro-phenyl)-1H-pyrazol-4-ylmethyl]-4-(5-iodo-pyridin-2-yl)-piperazine, 1-(4-Chloro-3-trifluoromethyl-phenyl)-4-[3-(4-fluoro-phenyl)-1H-pyrazol-4-ylmethyl]-piperazine, 1-[3-(4-Fluoro-phenyl)-1H-pyrazol-4-ylmethyl]-4-(3-trifluoromethyl-phenyl)-piperazine, 1-(4-Bromo-phenyl)-4-[3-(4-fluoro-phenyl)-1H-pyrazol-4-ylmethyl]-piperazine, 4-{4-[3-(4-Fluoro-phenyl)-1H-pyrazol-4-ylmethyl]-piperazin-1-yl}-phenol, 6-{4-[3-(4-Fluoro-phenyl)-1H-pyrazol-4-ylmethyl]-piperazin-1-yl}-pyridin-3-ylamine, 1-(3,4-Dimethyl-phenyl)-4-[3-(4-fluoro-phenyl)-1H-pyrazol-4-ylmethyl]-piperazine, 1-(2-Fluoro-phenyl)-4-[3-(4-fluoro-phenyl)-1H-pyrazol-4-ylmethyl]-piperazine, 6-{4-[3-(4-Fluoro-phenyl)-1H-pyrazol-4-ylmethyl]-piperazin-1-yl 4-nicotinic acid, 4-[3-(4-Fluoro-phenyl)-1H-pyrazol-4-ylmethyl]-2-methyl-1-(5-trifluoromethyl-pyridin-2-yl)-piperazine, 1-[3-(4-Fluoro-phenyl)-1H-pyrazol-4-ylmethyl]-2-methyl-4-(5-trifluoromethyl-pyridin-2-yl)-piperazine, 1-[3-(4-Fluoro-phenyl)-1H-pyrazol-4-ylmethyl]-4-(5-methyl-pyridin-2-yl)-piperazine, 1-(3-Chloro-pyridin-2-yl)-4-[3-(4-fluoro-phenyl)-1H-pyrazol-4-ylmethyl]-piperazine, 2-{4-[3-(4-Fluoro-phenyl)-1H-pyrazol-4-ylmethyl]-piperazin-1-yl}-isonicotinonitrile, 2-Fluoro-5-{4-[4-(5-trifluoromethyl-pyridin-2-yl)-piperazin-1-ylmethyl]-1H-pyrazol-3-yl}-benzonitrile, 2-Fluoro-5-{4-[4-(5-trifluoromethyl-pyridin-2-yl)-piperazin-1-ylmethyl]-1H-pyrazol-3-yl}-benzamide, 4-{4-[4-(5-Trifluoromethyl-pyridin-2-yl)-[1,4]diazepan-1-ylmethyl]-1H-pyrazol-3-yl}-benzonitrile, 2-Fluoro-5-{4-[4-(5-trifluoromethyl-pyridin-2-yl)-piperazin-1-ylmethyl]-1H-pyrazol-3-yl}-benzylamine, (2-Fluoro-5-{4-[4-(5-trifluoromethyl-pyridin-2-yl)-piperazin-1-ylmethyl]-1H-pyrazol-3-yl}-benzyl)-dimethyl-amine, N-(2-Fluoro-5-{4-[4-(5-trifluoromethyl-pyridin-2-yl)-piperazin-1-ylmethyl]-1H-pyrazol-3-yl}-benzyl)-formamide, 1-(4-Chloro-phenyl)-4-[3-(4-fluoro-phenyl)-1H-pyrazol-4-ylmethyl]-piperazine, 1-[3-(4-Fluoro-phenyl)-1H-pyrazol-4-ylmethyl]-4-(4-methoxy-phenyl)-piperazine, 1-[3-(4-Fluoro-phenyl)-1H-pyrazol-4-ylmethyl]-4-p-tolyl-piperazine, 1-(3-Chloro-phenyl)-4-[3-(4-fluoro-phenyl)-1H-pyrazol-4-ylmethyl]-piperazine, 1-(2,4-Difluoro-phenyl)-4-[3-(4-fluoro-phenyl)-1H-pyrazol-4-ylmethyl]-piperazine, 1-(3,4-Dichloro-phenyl)-4-[3-(4-fluoro-phenyl)-1H-pyrazol-4-ylmethyl]-piperazine, 1-(2,3-Dichloro-phenyl)-4-[3-(4-fluoro-phenyl)-1H-pyrazol-4-ylmethyl]-piperazine, 1-(3,5-Dichloro-phenyl)-4-[3-(4-fluoro-phenyl)-1H-pyrazol-4-ylmethyl]-piperazine, 1-(2,3-Dimethyl-phenyl)-4-[3-(4-fluoro-phenyl)-1H-pyrazol-4-ylmethyl]-piperazine, 1-(2,4-Dimethyl-phenyl)-4-[3-(4-fluoro-phenyl)-1H-pyrazol-4-ylmethyl]-piperazine, 4-{4-[4-(5-Chloro-pyridin-2-yl)-piperazin-1-ylmethyl]-1H-pyrazol-3-yl}-benzonitrile, 2-{4-[3-(4-Cyano-phenyl)-1H-pyrazol-4-ylmethyl]-piperazin-1-yl}-isonicotinonitrile, 4-{4-[4-(4-Chloro-3-trifluoromethyl-phenyl)-piperazin-1-ylmethyl]-1H-pyrazol-3-yl}-benzonitrile, 4-{4-[4-(3,4-Dimethyl-phenyl)-piperazin-1-ylmethyl]-1H-pyrazol-3-yl}-benzonitrile, 1-[3-(4-Fluoro-phenyl)-1H-pyrazol-4-ylmethyl]-4-(4-methyl-pyridin-2-yl)-piperazine, 1-(2,4-Dichloro-phenyl)-4-[3-(4-fluoro-phenyl)-1H-pyrazol-4-ylmethyl]-piperazine, 1-(4-Chloro-2-fluoro-phenyl)-4-[3-(4-fluoro-phenyl)-1H-pyrazol-4-ylmethyl]-piperazine, 2-Cyano-5-{4-[4-(5-trifluoromethyl-pyridin-2-yl)-piperazin-1-ylmethyl]-1H-pyrazol-3-yl}-benzamide, 2-Cyano-5-{4-[4-(5-trifluoromethyl-pyridin-2-yl)-[1,4]diazepan-1-ylmethyl]-1H-pyrazol-3-yl}-benzamide, 2-Cyano-N-methyl-5-{4-[4-(5-trifluoromethyl-pyridin-2-yl)-[1,4]diazepan-1-ylmethyl]-1H-pyrazol-3-yl}-benzamide, 4-{4-[3-Methyl-4-(5-trifluoromethyl-pyridin-2-yl)-piperazin-1-ylmethyl]-1H-pyrazol-3-yl}-benzonitrile, 4-{4-[3-Methyl-4-(5-trifluoromethyl-pyridin-2-yl)-piperazin-1-ylmethyl]-1H-pyrazol-3-yl}-benzonitrile, 4-{4-[2-Methyl-4-(5-trifluoromethyl-pyridin-2-yl)-piperazin-1-ylmethyl]-1H-pyrazol-3-yl}-benzonitrile, 4-{4-[5-(5-Trifluoromethyl-pyridin-2-yl)-2,5-diaza-bicyclo[2.2.1]hept-2-ylmethyl]-1H-pyrazol-3-yl}-benzonitrile, 4-{4-[2-Methyl-4-(5-trifluoromethyl-pyridin-2-yl)-piperazin-1-ylmethyl]-1H-pyrazol-3-yl}-benzonitrile, 4-{4-[3,5-Dimethyl-4-(5-trifluoromethyl-pyridin-2-yl)-piperazin-1-ylmethyl]-1H-pyrazol-3-yl}-benzonitrile, 4-{4-[4-(6-Trifluoromethyl-pyridin-3-yl)-piperazin-1-ylmethyl]-1H-pyrazol-3-yl}-benzonitrile, 4-{4-[4-(5-Trifluoromethyl-pyridin-2-yl)-piperazin-1-ylmethyl]-1H-pyrazol-3-yl}-phenol, 1-[3-(4-Bromo-phenyl)-1H-pyrazol-4-ylmethyl]-4-(5-trifluoromethyl-pyridin-2-yl)-piperazine, Ethyl-carbamic acid 4-{4-[4-(5-trifluoromethyl-pyridin-2-yl)-piperazin-1-ylmethyl]-1H-pyrazol-3-yl}-phenyl ester, 1-[3-(4-Imidazol-1-yl-phenyl)-1H-pyrazol-4-ylmethyl]-4-(5-trifluoromethyl-pyridin-2-yl)-piperazine, 2-Methyl-4-{3-[4-(1H-pyrazol-4-yl)-phenyl]-1H-pyrazol-4-ylmethyl}-1-(5-trifluoromethyl-pyridin-2-yl)-piperazine, 4-{4-[3,3-Dimethyl-4-(5-trifluoromethyl-pyridin-2-yl)-piperazin-1-ylmethyl]-1H-pyrazol-3-yl}-benzonitrile, 4-{4-[2,5-Dimethyl-4-(5-trifluoromethyl-pyridin-2-yl)-piperazin-1-ylmethyl]-1H-pyrazol-3-yl}-benzonitrile, Ethyl-carbamic acid 4-{4-[3-methyl-4-(5-trifluoromethyl-pyridin-2-yl)-piperazin-1-ylmethyl]-1H-pyrazol-3-yl}-phenyl ester, 4-{4-[3-Ethyl-4-(5-trifluoromethyl-pyridin-2-yl)-piperazin-1-ylmethyl]-1H-pyrazol-3-yl}-benzonitrile, 1-Ethyl-3-(4-{4-[3-methyl-4-(5-trifluoromethyl-pyridin-2-yl)-piperazin-1-ylmethyl]-1H-pyrazol-3-yl}-phenyl)-urea, Methyl-carbamic acid 4-{4-[4-(5-trifluoromethyl-pyridin-2-yl)-piperazin-1-ylmethyl]-1H-pyrazol-3-yl}-phenyl ester, (4-{4-[3-Methyl-4-(6-trifluoromethyl-pyridin-3-yl)-piperazin-1-ylmethyl]-1H-pyrazol-3-yl}-phenyl)-acetonitrile, 2-(4-{4-[3-Methyl-4-(6-trifluoromethyl-pyridin-3-yl)-piperazin-1-ylmethyl]-1H-pyrazol-3-yl}-phenyl)-acetamide, Dimethyl-(5-{4-[3-methyl-4-(5-trifluoromethyl-pyridin-2-yl)-piperazin-1-ylmethyl]-1H-pyrazol-3-yl}-pyridin-2-yl)-amine, (4-{4-[3-Methyl-4-(6-trifluoromethyl-pyridin-3-yl)-piperazin-1-ylmethyl]-1H-pyrazol-3-yl}-phenyl)-acetic acid, Isopropyl-carbamic acid 4-{4-[3-methyl-4-(5-trifluoromethyl-pyridin-2-yl)-piperazin-1-ylmethyl]-1H-pyrazol-3-yl}-phenyl ester, Phenyl-carbamic acid 4-{4-[3-methyl-4-(5-trifluoromethyl-pyridin-2-yl)-piperazin-1-ylmethyl]-1H-pyrazol-3-yl}-phenyl ester, 5-{4-[3-Methyl-4-(5-trifluoromethyl-pyridin-2-yl)-piperazin-1-ylmethyl]-1H-pyrazol-3-yl}-pyridine-2-carbonitrile, 6-{4-[3-Methyl-4-(5-trifluoromethyl-pyridin-2-yl)-piperazin-1-ylmethyl]-1H-pyrazol-3-yl}-nicotinonitrile, 2-(5-{4-[3-Methyl-4-(5-trifluoromethyl-pyridin-2-yl)-piperazin-1-ylmethyl]-1H-pyrazol-3-yl}-pyridin-2-yl)-acetamide, carbamic, 5-{4-[3-methyl-4-(5-trifluoromethyl-pyridin-2-yl)-piperazin-1-ylmethyl]-1H-pyrazol-3-yl}-pyridin-2-yl ester, (S)-methyl 4-((3-(4-cyanophenyl)-1H-pyrazol-4-yl)methyl)-1-(5-(trifluoromethyl)-pyridin-2-yl)piperazine-2-carboxylate; (S)-4-((3-(4-cyanophenyl)-1H-pyrazol-4-yl)methyl)-1-(5-(trifluoromethyl)pyridin-2-yl)piperazine-2-carboxylic acid; (S)-4-(4-((3-(methoxymethyl)-4-(5-(trifluoromethyl)pyridin-2-yl)piperazin-1-yl)methyl)-1H-pyrazol-3-yl)benzonitrile; (R)-2-(4-(4-((3-methyl-4-(5-(trifluoromethyl)pyridin-2-yl)piperazin-1-yl)methyl)-1H-pyrazol-3-yl)phenylamino)ethanol; (R)-5-(4-(4-((3-methyl-4-(5-(trifluoromethyl)pyridin-2-yl)piperazin-1-yl)methyl)-1H-pyrazol-3-yl)phenyl)isoxazole; (R)-4-((3-(4-(1H-pyrrol-1-yl)phenyl)-1H-pyrazol-4-yl)methyl)-2-methyl-1-(5-(trifluoromethyl)pyridin-2-yl)piperazine; (R)-2-methyl-4-((3-(4-(methylsulfonyl)phenyl)-1H-pyrazol-4-yl)methyl)-1-(5-(trifluoromethyl)pyridin-2-yl)piperazine; (R)-N-(4-(4-((3-methyl-4-(5-(trifluoromethyl)pyridin-2-yl)piperazin-1-yl)methyl)-1H-pyrazol-3-yl)phenylsulfonyl)acetamide; (R)-4-(4-((3-methyl-4-(5-(trifluoromethyl)pyridin-2-yl)piperazin-1-yl)methyl)-1H-pyrazol-3-yl)benzoic acid; (R)-4-((3-(4-(1H-tetrazol-5-yl)phenyl)-1H-pyrazol-4-yl)methyl)-2-methyl-1-(5-(trifluoromethyl)pyridin-2-yl)piperazine; (R)-4-((3-(4-((1H-tetrazol-5-yl)methyl)phenyl)-1H-pyrazol-4-yl)methyl)-2-methyl-1-(5-(trifluoromethyl)pyridin-2-yl)piperazine; (R)-2-(5-(4-((3-methyl-4-(5-(trifluoromethyl)pyridin-2-yl)piperazin-1-yl)methyl)-1H-pyrazol-3-yl)pyridin-2-ylamino)ethanol; (R)-2,2′-(5-(4-((3-methyl-4-(5-(trifluoromethyl)pyridin-2-yl)piperazin-1-yl)methyl)-1H-pyrazol-3-yl)pyridin-2-ylazanediyl)diethanol; (S)-4-(4-((3-(trifluoromethyl)-4-(5-(trifluoromethyl)pyridin-2-yl)piperazin-1-yl)methyl)-1H-pyrazol-3-yl)benzonitrile; (R)-4-{4-[3-Trifluoromethyl-4-(5-trifluoromethyl-pyridin-2-yl)-piperazin-1-ylmethyl]-1H-pyrazol-3-yl}-benzonitrile; (S)-4-(4-((2-(trifluoromethyl)-4-(5-(trifluoromethyl)pyridin-2-yl)piperazin-1-yl)methyl)-1H-pyrazol-3-yl)benzonitrile; (R)-4-(4-((2-(trifluoromethyl)-4-(5-(trifluoromethyl)pyridin-2-yl)piperazin-1-yl)methyl)-1H-pyrazol-3-yl)benzonitrile; (R)-4-(4-((2-(fluoromethyl)-4-(5-(trifluoromethyl)pyridin-2-yl)piperazin-1-yl)methyl)-1H-pyrazol-3-yl)benzonitrile; (S)-4-(4-((2-(fluoromethyl)-4-(5-(trifluoromethyl)pyridin-2-yl)piperazin-1-yl)methyl)-1H-pyrazol-3-yl)benzonitrile; 4-(4-((1-(5-(trifluoromethyl)pyridin-2-yl)piperidin-4-yl)methyl)-1H-pyrazol-3-yl)benzonitrile; 4-(4-((4-(5-(trifluoromethyl)pyridin-2-yl)piperidin-1-yl)methyl)-1H-pyrazol-3-yl)benzonitrile; (R)-5-(4-(4-((3-methyl-4-(5-(trifluoromethyl)pyridin-2-yl)piperazin-1-yl)methyl)-1H-pyrazol-3-yl)phenyl)oxazole; (R)-4-((3-(4-(1H-pyrazol-1-yl)phenyl)-1H-pyrazol-4-yl)methyl)-2-methyl-1-(5-(trifluoromethyl)pyridin-2-yl)piperazine; (R)-4-((3-(4-(1H-1,2,4-triazol-1-yl)phenyl)-1H-pyrazol-4-yl)methyl)-2-methyl-1-(5-(trifluoromethyl)pyridin-2-yl)piperazine; (R)-2-(4-(4-((3-methyl-4-(5-(trifluoromethyl)pyridin-2-yl)piperazin-1-yl)methyl)-1H-pyrazol-3-yl)phenylamino)acetic acid; (R)-N-methyl-4-(4-((3-methyl-4-(5-(trifluoromethyl)pyridin-2-yl)piperazin-1-yl)methyl)-1H-pyrazol-3-yl)benzenesulfonamide; (R)-1-(4-(4-((3-methyl-4-(5-(trifluoromethyl)pyridin-2-yl)piperazin-1-yl)methyl)-1H-pyrazol-3-yl)phenyl)-1H-pyrrole-2-carbonitrile; 4-(4-((3-(5-(trifluoromethyl)pyridin-2-yl)-3,8-diazabicyclo[3.2.1]octan-8-yl)methyl)-1H-pyrazol-3-yl)benzonitrile; 4-(4-((8-(5-(trifluoromethyl)pyridin-2-yl)-3,8-diazabicyclo[3.2.1]octan-3-yl)methyl)-1H-pyrazol-3-yl)benzonitrile; (R)-2-methyl-4-((3-(4-(2-methyl-1H-imidazol-1-yl)phenyl)-1H-pyrazol-4-yl)methyl)-1-(5-(trifluoromethyl)pyridin-2-yl)piperazine; (R)-2-methyl-4-((3-(4-(5-methyl-1H-imidazol-1-yl)phenyl)-1H-pyrazol-4-yl)methyl)-1-(5-(trifluoromethyl)pyridin-2-yl)piperazine; (R)-2-methyl-4-((3-(4-(4-methyl-1H-imidazol-1-yl)phenyl)-1H-pyrazol-4-yl)methyl)-1-(5-(trifluoromethyl)pyridin-2-yl)piperazine; (R)-N-(2-(5-(4-((3-methyl-4-(5-(trifluoromethyl)pyridin-2-yl)piperazin-1-yl)methyl)-1H-pyrazol-3-yl)pyridin-2-ylamino)ethyl)acetamide; (R)-N1-(5-(4-((3-methyl-4-(5-(trifluoromethyl)pyridin-2-yl)piperazin-1-yl)methyl)-1H-pyrazol-3-yl)pyridin-2-yl)ethane-1,2-diamine; (R)-4-(5-(4-((3-methyl-4-(5-(trifluoromethyl)pyridin-2-yl)piperazin-1-yl)methyl)-1H-pyrazol-3-yl)pyridin-2-yl)morpholino; (R)-5-(4-((3-methyl-4-(5-(trifluoromethyl)pyridin-2-yl)piperazin-1-yl)methyl)-1H-pyrazol-3-yl)-N-(2-(piperidin-1-yl)ethyl)pyridin-2-amine; (R)-4-(5-(4-((3-methyl-4-(5-(trifluoromethyl)pyridin-2-yl)piperazin-1-yl)methyl)-1H-pyrazol-3-yl)pyridin-2-yl)piperazin-2-one; (R)-2-hydroxy-4-(4-((3-methyl-4-(5-(trifluoromethyl)pyridin-2-yl)piperazin-1-yl)methyl)-1H-pyrazol-3-yl)benzoic acid; 1-(5-(4-(((R)-3-methyl-4-(5-(trifluoromethyl)pyridin-2-yl)piperazin-1-yl)methyl)-1H-pyrazol-3-yl)pyridin-2-yl)pyrrolidin-3-ol; 4-(4-((7-(5-(trifluoromethyl)pyridin-2-yl)-4,7-diazaspiro[2.5]octan-4-yl)methyl)-1H-pyrazol-3-yl)benzonitrile; 4-(4-((4-(5-(trifluoromethyl)pyridin-2-yl)-4,7-diazaspiro[2.5]octan-7-yl)methyl)-1H-pyrazol-3-yl)benzonitrile; 1-(5-(4-(((R)-3-methyl-4-(5-(trifluoromethyl)pyridin-2-yl)piperazin-1-yl)methyl)-1H-pyrazol-3-yl)pyridin-2-ylamino)propan-2-ol; ((S)-1-(5-(4-(((R)-3-methyl-4-(5-(trifluoromethyl)pyridin-2-yl)piperazin-1-yl)methyl)-1H-pyrazol-3-yl)pyridin-2-yl)pyrrolidin-2-yl)methanol; (R)-1-(5-(4-(((R)-3-methyl-4-(5-(trifluoromethyl)pyridin-2-yl)piperazin-1-yl)methyl)-1H-pyrazol-3-yl)pyridin-2-ylamino)propan-2-ol; (S)-1-(5-(4-(((R)-3-methyl-4-(5-(trifluoromethyl)pyridin-2-yl)piperazin-1-yl)methyl)-1H-pyrazol-3-yl)pyridin-2-ylamino)propan-2-ol; (R)-2-(5-(4-((3-methyl-4-(5-(trifluoromethyl)pyridin-2-yl)piperazin-1-yl)methyl)-1H-pyrazol-3-yl)pyridin-2-yloxy)ethanol; and (R)-1-(5-(4-((3-methyl-4-(5-(trifluoromethyl)pyridin-2-yl)piperazin-1-yl)methyl)-1H-pyrazol-3-yl)pyridin-2-yl)azetidin-3-ol.

8. A method for modulating B lymphocyte development and function in a subject for the treatment of autoimmune diseases, the method comprising administering to the subject a pharmaceutical composition comprising an effective amount of an agent which modulates the kinase activity or cellular level of an ITPKb molecule; thereby modulating B lymphocyte differentiation and function in a subject.

9. The method of claim 8 wherein the agent down-regulates the cellular level of the ITPKb molecule.

10. The method of claim 9 wherein the agent is a compound of claim 1.

11. The method of claim 10 wherein the agent inhibits the kinase activity of the ITPKb molecule.

12. The method of claim 11 wherein the subject is human and the ITPKb molecule is human ITPK\tilde{b}

13. The method of claim 12 in which the autoimmune disease is selected from rheumatoid arthritis and systemic lupus erythematosus.

14. The method of claim 12 wherein the subject suffers from B cell lymphoma.
